SEQUENCE LISTING (14 sequences)

SEQ ID NO: 1 - DNA, 22 bp, Artificial Sequence
  synthetic oligonucleotide
cggccgaaggaacctgacccag

SEQ ID NO: 2 - DNA, 23 bp, Artificial Sequence
  synthetic oligonucleotide
ccgttgggtcactcccaaggtcg

SEQ ID NO: 3 - DNA, 20 bp, Artificial Sequence
  synthetic oligonucleotide
aagtaggacccgaggcactg

SEQ ID NO: 4 - DNA, 20 bp, Artificial Sequence
  synthetic oligonucleotide
gtctggcgaagaaggagaag

SEQ ID NO: 5 - DNA, 19 bp, Artificial Sequence
  synthetic oligonucleotide
tggaggaccagaggccccc

SEQ ID NO: 6 - DNA, 22 bp, Artificial Sequence
  synthetic oligonucleotide
cgtccggaggactattagcagg

SEQ ID NO: 7 - DNA, 18 bp, Artificial Sequence
  synthetic oligonucleotide
gagcagacaggccaaccg

SEQ ID NO: 8 - DNA, 18 bp, Artificial Sequence
  synthetic oligonucleotide
cggactgcgtctcaggaa

SEQ ID NO: 9 - DNA, 19 bp, Artificial Sequence
  synthetic oligonucleotide
tggaggaccagaggccccc

SEQ ID NO: 10 - DNA, 24 bp, Artificial Sequence
  synthetic oligonucleotide
tgtccgaaggactattagtaggac

SEQ ID NO: 11 - DNA, 20 bp, Artificial Sequence
  synthetic oligonucleotide
ggtggaagtggtccgcatcg

SEQ ID NO: 12 - DNA, 22 bp, Artificial Sequence
  synthetic oligonucleotide
aacgatttctagtcacctcccg

SEQ ID NO: 13 - DNA, 1549 bp, Homo sapiens
  CDS (376)...(1239)
aaaccctctgtaaagtaacagaagttagaaggggaaatgtcgcctctctgaagattacccaaagaaaaagtgatttgtcattgctttatagactgtaagaagagaacatctcagaagtggagtcttaccctgaaatcaaaggatttaaagaaaaagtggaatttttcttcagcaagctgtgaaactaaatccacaacctttggagacccaggaacaccctccaatctctgtgtgttttgtaaacatcactggagggtcttctacgtgagcaattggattgtcatcagccctgcctgttttgcacctgggaagtgccctggtcttacttgggtccaaattgttggctttcacttttgaccctaagcatctgaagccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaaagatctgaaggtagcctccgtcatctcttctgggatacatggatcgtggggatcatgaggcattcttcccttaacaaatttaagctgttttacccactacctcaccttcttaaaaacctctttcagattaagctgaacagttacaagatggctggcatccctctcctttctccccatatgcaatttgcttaatgtaacctcttcttttgccatgtttccattctgccatcttgaattgtcttgtcagccaattcattatctattaaacactaatttgag

SEQ ID NO: 14 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

(19) United States
(12) Patent Application Publication
(10) Pub. No.: US 2002/0006413 A1
(43) Pub. Date: Jan. 17, 2002

(54) Genetically engineered tumor cell vaccines

(76) Inventors: Robert E. Sobol, La Jolla, CA (US); Daniel L. Shawler, San Diego, CA (US); Richard M. Bartholomew, San Diego, CA (US); Dennis J. Carlo, Rancho Santa Fe, CA (US); Daniel P. Gold, Del Mar, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/772,102

(22) Filed: Jan. 26, 2001

Related U.S. Application Data
(60) Provisional application No. 60/178,498, filed on Jan. 27, 2000.
(60) Provisional application No. 60/185,335, filed on Feb. 28, 2000.

(51) Int. Cl. A61K 48/00

(52) U.S. Cl. 424/277.1; 424/93.21

(57) ABSTRACT

The invention provides a composition for stimulating an immune response in a patient having an adenocarcinoma, including a patient having colorectal cancer, containing allogeneic tumor cells and a physiologically acceptable carrier. The allogeneic tumor cells can be SW620, COLO 205, or SW403 cells. The invention composition can also contain an allogeneic cell expressing a cytokine. An allogeneic tumor cell can additionally express CD80. The invention additionally provides a method of stimulating an immune response in a patient having an adenocarcinoma, including a patient having colorectal cancer, by administering to the patient one or more allogeneic tumor cells, wherein the allogeneic cell stimulates an immune response to autologous tumor cells in the patient.

This application claims the benefit of priority of U.S. Ser. No. 60/178,498, filed Jan. 27, 2000, and Ser. No. 60/185,335, filed Feb. 28, 2000, each of which the entire contents is incorporated herein by reference.

This invention was made with government support under grant number AG0353 and CA67814 awarded by the National Institutes of Health. The government has certain rights in the inventions.

### BACKGROUND OF THE INVENTION

The present invention relates generally to cancer therapy and more specifically to tumor vaccines.

Colorectal carcinoma is one of the most common cancers in the United States and Europe, with an annual incidence of greater than 150,000 in the U.S. Most patients are treated with tumor resection and do not have clinically detectable tumor following surgery. However, the majority of patients have microscopic metastases and eventually relapse with clinically overt disease in the liver or abdominal cavity.

The relative chemotherapy resistance of these recurrent colon cancer metastases further emphasizes the need for new treatment modalities, such as adjuvant immunotherapy. Recent scientific advances in the biology of the immune system such as the identification of immunostimulatory genes, combined with improvements in the ability to modify gene expression, have fostered a new era of tumor immunotherapy (Borden and Sondel, Cancer, 65:800-814 (1990); Rosenberg et al., Ann. Intern. Med., 108:853-864 (1988)).

The large number of patients and the small tumor burden following surgical resection makes colorectal carcinoma an attractive candidate for adjuvant immunotherapy trials. It is generally acknowledged that immunotherapies are likely to be most effective when the tumor burden is low. In this regard, the immunomodulator Levamisole is currently approved for the treatment of patients with Duke's C tumors (metastases to abdominal lymph nodes). In addition, encouraging results have been obtained with an autologous tumor vaccine as an adjuvant therapy following tumor resection.

Vaccinations with tumor cells genetically engineered to express immuno-stimulatory cytokines have resulted in significant anti-tumor immune responses in several animal tumor models (Fakhrai et al., Human Gene Therapy, 6:591-601 (1995); Fearon et al., Cell, 60:387-403 (1990); Gansbacher et al., J. Exp. Med., 172:1217-1223 (1990); Tepper et al., Cell, 57:503-512 (1989)). The effects of IL-2 gene transfer in human subjects has been evaluated (Sobol et al., Gene Therapy, 2:164-167 (1995); Sobol et al., Clin Cancer Res, 5:2359-2365 (1999)). A Phase I clinical trial was performed in colorectal carcinoma patients consisting of immunizations with a mixture of irradiated, autologous tumor cells and autologous fibroblasts genetically modified to express the gene for IL-2. It was demonstrated that patients with colorectal cancer have low frequencies of anti-tumor cytotoxic T cell (CTL) precursors that are increased by this form of IL-2 gene therapy.

It is becoming increasingly clear that co-stimulatory second signals, such as co-ligation of auxiliary molecules, are critical for generating T cell mediated anti-tumor immunity (Mondino and Jenkins, J. Leukocyte Biol., 55:805-815 (1994); June et al., Immunology Today, 11:211-216 (1990)). In contrast, antigen recognition in the absence of these second signals can lead to tolerance or anergy (Mondino and Jenkins, J. Leukocyte Biol., 55:805-815 (1994); June et al., Immunology Today, 11:211-216 (1990)). Vaccination with MHC-matched tumor cells genetically modified to express the co-stimulatory molecule CD80 have demonstrated synergistic effects with IL-2 gene transfer in generating efficacious anti-tumor immunity in animal tumor models (Baskar et al., J. Exp. Med., 181:619-629 (1995)).

Although the use of autologous tumor cells combined with immuno-stimulatory cytokines is a promising therapy, it requires that primary fibroblasts and colon tumor cultures be established for each individual patient. Therefore, such methods are limited by the ability to obtain cells and culture a sufficient number of autologous fibroblasts and colon tumor cells from each patient. Furthermore, even in those patients from which autologous cells are successfully cultured, the initiation of such treatments must be delayed until a sufficient quantity of cells for therapy is obtained.

Thus, there exists a need for therapeutic methods that enhance anti-tumor responses to autologous tumors that can be efficiently applied to a variety of colon cancer patients. The present invention satisfied this need and provides related advantages as well.

### SUMMARY OF THE INVENTION

The invention provides a composition for stimulating an immune response in a patient having an adenocarcinoma containing an allogeneic tumor cell and a physiologically acceptable carrier. The adenocarcinoma can be, for example, colon, breast, lung or prostate adenocarcinoma. The allogeneic tumor cell can be a SW620 cell, COLO 205 cell, or SW403 cell. The invention also provides a composition containing allogeneic tumor cells and an allogeneic cell expressing a cytokine. The invention additionally provides a method of stimulating an immune response in a patient having colorectal cancer by administering to the patient one or more allogeneic tumor cells, wherein at least one of the allogeneic tumor cells is selected from the group consisting of SW620, COLO 205, and SW403 and wherein the allogeneic cell stimulates an immune response to autologous tumor cells in the patient. The method can further include an allogeneic cell such as a fibroblast genetically modified to express a cytokine.

### BRIEF DESCRIPTION OF THE DRAWINGS

FIG. 1 shows TGF-β secretion by colon carcinoma cell lines. The top panels (A and B) show TGF-β secretion by fresh colon carcinoma cell cultures. The bottom panels (C and D) show TGF-β secretion by established colon carcinoma cell lines. TGF-β1 secretion is shown in the left panels (A and C) and TGF-β2 secretion is shown in the right panels (B and D).

FIG. 2 shows HLA-A2-restricted cross-reactive cytotoxicity induced by stimulation with SW620. A CTL clone was generated in vitro by limiting dilution stimulation of HLA-A2-positive PBMC by the HLA-A2 positive colon carcinoma cell line SW620 and then tested for cytotoxicity against four cell lines using a standard chromium-release assay. Colon carcinoma line SW620 (●); colon tumor GT53T (▴); normal skin fibroblast line GT53F (Δ); colon carcinoma line HT-29 (∇).

FIG. 3 shows enhancement of cytolytic activity by CD80-expressing colon carcinoma cells. CTL were generated in vitro by stimulating PBMC with parental or CD80 gene modified SW620 (panel A) or COLO 205 (panel B). CD80-expressing clones of SW620 and COLO 205 (●); the parental SW620 and COLO 205 lines (∘).

FIG. 4 shows SW620 lysis by anti-p53 CTL A2 264, clone 15. CTL A2 264 clone 15 (●); HIV pol 9K antigen negative control (▴).

FIG. 5 shows reactivity of T cell clones derived from patients immunized with allogeneic tumor cells.

FIG. 6 shows construction of pKIM-kan plasmid vector.

FIG. 7 shows construction of pKIM-kan/tIL-2 plasmid vector.

FIG. 8 shows construction of pKIM-kan/B7.1 plasmid vector.

### DETAILED DESCRIPTION OF THE INVENTION

The invention provides compositions and methods for stimulating an immune response in a patient having an adenocarcinoma using allogeneic tumor cells. The compositions and methods of the invention are particularly useful for stimulating an immune response in a patient having colorectal cancer. The allogeneic tumor cells can be genetically modified to enhance an immune response. The allogeneic vaccine can further include an allogeneic cell genetically modified to express a cytokine. The invention also provides methods of stimulating an immune response in a patient having an adenocarcinoma, including a patient having colorectal cancer, by administering one or more allogeneic tumor cells, wherein the allogeneic tumor cell stimulates an immune response to an autologous tumor cell in the patient. The methods of the invention can further include administering an allogeneic cell genetically modified to express a cytokine.

The methods of the invention are advantageous in that they utilize one or more allogeneic tumor cells expressing antigens that are expressed in a patient having an adenocarcinoma, for example, colon, breast, lung or prostate adenocarcinoma, thereby stimulating an immune response to the antigens. The use of allogeneic tumor cells provides a generic source of antigen that can be administered to a variety of patients, in contrast to using autologous tumor cells, which must be isolated from each individual patient. The methods of the invention are advantageous in that the allogeneic cells are suitable as a cancer vaccine and can stimulate an immune response against autolgous tumor cells of a cancer patient.

As used herein, an “autologous cell” refers to a cell derived from a specific individual. In methods of the invention, the specific individual from which an autologous cell is derived refers to an individual administered an allogeneic vaccine of the invention. As used herein, an “autologous tumor cell” refers to a cell derived from a tumor in such an individual.

As used herein, an “allogeneic cell” refers to a cell that is not derived from the individual administered an invention vaccine, that is, has a different genetic constitution than the individual. An allogeneic cell is generally obtained from the same species as the individual administered an invention vaccine. In particular, a human allogeneic cell can be used to stimulate an immune response in a human individual having cancer (see Examples). As used herein, an “allogeneic tumor cell” refers to a tumor cell that is not derived from the individual to which the allogeneic cell is to be administered. An allogeneic tumor cell expresses at least one tumor antigen that is common to an autologous tumor cell in a patient. Generally, the allogeneic cell is derived from a similar type of tumor as that being treated in the patient. For example, as disclosed herein, a patient being treated for colorectal cancer can be administered an allogeneic tumor cell derived from a colorectal tumor. Exemplary allogeneic tumor cells include the SW620, COLO 205, and SW403 cell lines described herein (see Examples I to III). Other exemplary tumor cells include, for example, GT23T, GT42T, GT45T, GT50T, GT53T, GT54T, GT56T, GT62T, GT64T, GT70T, GT71T, GT72T, HCT-15, HCT-116, SW480, WiDr, COLO 320DM, COLO 320HSR, DLD-1, COLO 201, LoVo, SW48, SW1116, SW837, SW948, SW1417, HCT-8 (HRT-18), NCI-H548, LS 180, LS 174T, LS1034, Caco-2, HT-29, SK-CO-1, SNU-C2A, NCI-H548, NCI-H742, NCI-H768, NCI-H854. Such allogeneic cell lines are available at the American Type Culture Collection (ATCC; Manassas, Va.), for example, ATCC numbers in parentheses: SW620(CCL-227), COLO205 (CCL-222), SW403 (CCL-230), HCT-15 (CCL-225), HCT-116 (CCL-247), SW480 (CCL-228), WiDr (CCL-218), COLO 320DM (CCL-220), COLO 320HSR (CCL-220.1), DLD-1 (CCL-221), COLO 201 (CCL-224), LoVo (CCL-229), SW48 (CCL-231), SW1116 (CCL-233), SW837 (CCL-235), SW948 (CCL-237), SW1417 (CCL-238), HCT-8 (HRT-18) (CCL-244), NCI-H548 (CCL-249), LS 180 (CL-187), LS 174T (CL-188), LS1034 (CRL-2158), Caco-2 (HTB-37), HT-29 (HTB-38), SK-CO-1 (HTB-39), SNU-C2A (CCL-250.1), SNU-C2B (CCL-250), NCI-H548 (CCL-249).

Although an allogeneic tumor cell can be derived from a colon tumor, the methods of the invention can also utilize an allogeneic cell expressing one or more tumor antigens. For example, an allogeneic cell can be engineered to express one or more tumor antigens specific for a particular tumor. For example, to treat a colon carcinoma, a cell can be genetically engineered to express tumor antigens expressed in a colorectal carcinoma. Exemplary tumor antigens suitable for an allogeneic tumor cell for treatment of a colorectal carcinoma include, for example, carcinoembryonic antigen (CEA), MUC-1, Ep-CAM, HER2/neu, p53, and MAGE, including MAGE 1, 2, 3, 4, 6 and 12. Additional tumor antigens can also be expressed in an allogeneic cell and used in an allogeneic vaccine of the invention. Additional tumor antigens can be identified using well known methods of screening for tumor antigens using, for example, tumor specific antibodies. Additional tumor antigens can be cloned into an allogeneic cell and expressed. Methods of genetically engineering a cell to express a particular gene is well known to those skilled in the art (see Example II and Sambrook et al., Molecular Cloning: A Laboratory Manual, 2nd ed., Cold Spring Harbor Press, Plainview, N.Y. (1989); Ausubel et al., Current Protocols in Molecular Biology (Supplement 47), John Wiley & Sons, New York (1999)).

In addition to colorectal cancer, an invention vaccine can be used to treat an individual having other types of cancers, in particular, patients having adenocarcinoma. Because many adenocarcinomas share antigens, as described in more detail below, an invention vaccine used to treat one type of adenocarcinoma can also be used to treat other types of adenocarcinomas if the tumors share antigens with the allogeneic tumor cell of an invention vaccine. Similarly, other types of tumors having shared antigens can be treated with an invention vaccine. As used herein, a “patient having an adenocarcinoma” refers to an individual having signs or symptoms associated with an adenocarcinoma. An adenocarcinoma is a malignant neoplasm of epithelial cells in glandular or glandlike pattern. Exemplary adenocarcinomas include those of colon, breast, lung, prostate, pancreas, kidney, endometrium, cervix, ovary, thyroid, or other glandular tissues.

As used herein, a “patient having colorectal cancer” refers to an individual having signs or symptoms associated with colorectal cancer. The major symptoms of colorectal cancer include rectal bleeding, abdominal pain and change in bowel habit. Colorectal cancer can be diagnosed by physical examination and selected use of laboratory or radiologic tests, including colonoscopy or double-contrast barium enema following signmoidoscopy, endoscopic ultrasonography, and/or histology of biopsy specimens. One skilled in the art can readily determine if an individual has signs or symptoms of colorectal cancer.

As used herein, an “immune response” refers to a measurable response to an antigen mediated by one or more cells of the immune system. An immune response can include a humoral or cellular response. As used herein, an immune response to an autologous tumor cell antigen refers to a measurable immune response to at least one antigen expressed on an autologous tumor cell. Similarly, an immune response to an autologous tumor cell refers to an immune response that is detectable and specific for an autologous tumor cell. As disclosed herein, use of an invention allogeneic vaccine in a colorectal carcinoma patient resulted in a detectable immune response to autologous tumor cells (see Example III).

As used herein, a “cytotoxic T lymphocyte response” or “CTL response” refers to an immune response in which cytotoxic T cells are activated. A CTL response includes the activation of precursor CTLs as well as differentiated CTLs. For example, as disclosed herein, administering a vaccine containing allogeneic colorectal carcinoma cell lines increased the frequency of precursor CTLs specific for tumor antigens of the allogeneic cell lines. The vaccine also stimulated the frequency of CTLs for autologous tumor cells (see Example III).

As used herein, a CTL response is intended to include any measurable CTL response for a particular antigen. Preferably, the CTL response includes at least one CTL that is specific for an antigen expressed on an autologous tumor cell. The level of CTL response can range from a modest response to an intermediate response as well as a strong CTL response. Even a modest response can be effective in treating a cancer patient if such treatment stimulates an immune response against autologous tumor cells in the patient.

As disclosed herein, an allogeneic tumor cell vaccine increased the frequency of precursor CTLs in a patient administered the vaccine (Example III). The allogeneic vaccine stimulated a 5- to 10-fold increase in the frequency of precursor CTLs. It is understood that any increase in CTL response is considered a stimulated CTL response so long as the CTL response is against at least one antigen associated with an autologous tumor in the patient.

As used herein, an exogenous cytokine refers to a cytokine that is administered to an individual. For example, an exogenous cytokine can be administered as a cytokine composition, or the cytokine can be administered as a cell that expresses a cytokine.

The allogeneic tumor cell vaccine of the invention can be administered with an allogeneic cell expressing a cytokine. The cytokine-expressing allogeneic cell can be a non-tumor cell such as a fibroblast or a tumor cell. For example, as disclosed herein, a cytokine-expressing allogeneic fibroblast cell genetically modified to express IL-2 was administered as a component of an allogeneic tumor cell vaccine (see Examples I to III). Cytokines useful in methods of the invention are those that enhance an immune response to a tumor antigen. Exemplary cytokines include interleukin-1 (IL-1), IL-2, IL-3, IL-4, IL-5, IL-6, gamma-interferon, and granulocyte macrophage-colony stimulating factor (GM-CSF). If desired, the cytokine can be expressed in various functional forms so long as the cytokine retains activity to enhance an immune response. For example, a cytokine such as GM-CSF can function in a soluble or membrane-bound form (see U.S. Pat. No. 5,891,432, issued Apr. 6, 1999). Particularly useful cytokines for use in an allogeneic tumor cell vaccine of the invention are IL-2 and GM-CSF. Methods for modifying cells to express a cytokine for stimulating an immune response are well known to those skilled in the art (see, for example, U.S. Pat. Nos. 5,674,486 and 5,681,562, both of which issued Oct. 28, 1997).

A cytokine-expressing allogeneic cell can be any carrier cell that provides a sufficient level of cytokine expression to enhance an immune response. As used herein, an enhanced immune response is any measurable increase in an immune response. Essentially any cell type that provides sufficient expression of a cytokine to enhance an immune response can be used in methods of the invention. Particularly useful allogeneic cells for expressing a cytokine include allogeneic fibroblast cells and allogeneic tumor cells. Methods of genetically modifying an allogeneic cell to express a cytokine are well known to those skilled in the art (Sambrook et al., supra, 1989; Ausubel et al., supra, 1999). For example, a fibroblast cell was genetically modified to express IL-2 (see Examples I to III).

Additionally, allogeneic tumor cells can be modified to express a cytokine. An allogeneic tumor cell expressing antigens common to a tumor in a patient can be genetically modified to express a cytokine. For example, in a colorectal cancer patient, an allogeneic colorectal cancer cell can be genetically modified to express a cytokine, including SW620, COLO 205, SW403, other colorectal cancer cells disclosed herein, or any allogeneic cell expressing antigens common to a tumor in a patient. If desired, the cytokine expressing tumor cell can be genetically modified with additional molecules useful for stimulating or enhancing an immune response, for example, CD80. The cytokine expressed in the allogeneic cell can be any cytokine that enchances an immune response, including those disclosed herein. Particularly useful cytokines for use in methods of the invention include IL-2 and GM-CSF. In the case where the cytokine is expressed in an allogeneic tumor cell, GM-CSF can be expressed in the membrane-bound form to enhance an immune response to tumor antigens of the allogeneic tumor cell.

As used herein, a physiologically acceptable carrier useful in invention vaccines refers to any of the well known components useful for immunization. The components of the physiological carrier are intended to facilitate or enhance an immune response to an antigen administered in a vaccine. The formulations can contain buffers to maintain a preferred pH range, salts or other components that present the antigen to an individual in a composition that stimulates an immune response to the antigen. The physiologically acceptable carrier can also contain one or more adjuvants that enhance the immune response to the antigen. Formulations can be administered subcutaneously, intramuscularly, intradermally, or in any manner acceptable for immunization.

As used herein, the term “adjuvant” refers to a substance which, when added to an immunogenic agent such as an allogeneic tumor cell, nonspecifically enhances or potentiates an immune response to the agent in the recipient host upon exposure to the mixture. Adjuvants can include, for example, oil-in-water emulsions, water-in oil emulsions, alum (aluminum salts), liposomes and microparticles, such as, polysytrene, starch, polyphosphazene and polylactide/polyglycosides. Adjuvants can also include, for example, squalene mixtures (SAF-I), muramyl peptide, saponin derivatives, mycobacterium cell wall preparations, monophosphoryl lipid A, mycolic acid derivatives, nonionic block copolymer surfactants, Quil A, cholera toxin B subunit, polyphosphazene and derivatives, and immunostimulating complexes (ISCOMs) such as those described by Takahashi et al. (1990) Nature 344:873-875. For veterinary use and for production of antibodies in animals, mitogenic components of Freund's adjuvant (both complete and incomplete) can be used. In humans, Incomplete Freund's Adjuvant (IFA) is a preferred adjuvant. Various appropriate adjuvants are well known in the art (see, for example, Warren and Chedid, CRC Critical Reviews in Immunology 8:83 (1988); Allison and Byars, in Vaccines: New Approaches to Immunological Problems, Ellis, ed., Butterworth-Heinemann, Boston (1992)). Additional adjuvants include, for example, bacille Calmett-Gúerin (BCG), DETOX (containing cell wall skeleton of Mycobacterium phlei (CWS) and monophosphoryl lipid A from Salmonella Minnesota (MPL)), and the like (see, for example, Hoover et al., J. Clin. Oncol., 11:390 (1993); Woodlock et al., J. Immunotherapy 22:251-259 (1999)).

Furthermore, a cytokine can also be used as an adjuvant to enhance an immune response, as described above and disclosed herein. In particular, the methods of the invention can advantageously use a vaccine containing allogeneic tumor cells and an allogeneic cell genetically modified to express a cytokine such as IL-2, GM-CSF, or other cytokines, as disclosed herein (see Examples I to III). The use of cytokine expressing cells allows enhancement of the immune response to antigens of the allogeneic tumor cells, as described below. It is understood that more than one cytokine can be administered, if desired, either directly administering one or more cytokines or administering cytokines as a cell expressing multiple cytokines or multiple cells expressing multiple cytokines, or combinations thereof.

The invention provides a composition for stimulating an immune response in a patient having an adenocarcinoma. For example, the invention provides a composition for stimulating an immune response in a patient having colorectal cancer. The composition contains one or more allogeneic tumor cells selected from the group consisting of SW620, COLO 205, and SW403 and a physiologically acceptable carrier. The invention also provides a composition containing SW620, COLO 205, and SW403. The invention further provides a composition containing one or more allogeneic tumor cells selected from the group consisting of SW620, COLO 205, and SW403, an allogeneic fibroblast cell genetically modified to express a cytokine such as IL-2 or GM-CSF, and a physiologically acceptable carrier. In addition, other allogeneic tumor cells, as disclosed herein, can be included in an invention composition for stimulating an immune response.

As disclosed herein, the allogeneic tumor cells can be genetically modified to express molecules that enhance an immune response. For example, the allogeneic cells can be modified to express CD80 (B7.1) (see Examples I and II). In one embodiment, the genetically modified cell is SW620 or COLO 205, or a combination of both cells genetically modified. As disclosed herein, SW620 and COLO 205 were genetically modified to express CD80 (B7.1) and functioned to stimulate a CTL response (see Examples I-III). Furthermore, as described above, the allogeneic tumor cells can be modified to express a cytokine.

The allogeneic tumor cells are administered at a dose sufficient to stimulate an immune response to one or more antigens of the allogeneic tumor cell that are common to an autologous tumor in a patient. One skilled in the art can readily determine an appropriate dose range for administering sufficient allogeneic tumor cells to elicit an immune response. Such a dose can be at least about 1×10² cells, about 1×10³ cells, about 1×10⁴ cells, about 1×10⁵ cells, about 1×10⁶ cells, about 1×10⁷ cells, about 1×10⁸ cells, about 1×10⁹ cells, or about 1×10¹⁰ cells, or more. For example, as disclosed herein, allogeneic tumor cells administered at a total dose of about 6×10⁷ cells was sufficient to stimulate a CTL response. If more than one allogeneic tumor cell is administered, each cell can be administered at an individual dose so that an appropriate total dose of cells is administered. As disclosed herein, an allogeneic tumor cell vaccine was administered as a mixture of about 2×10⁷ cells of each of SW620, COLO 205, and SW403 (Example III).

The invention also provides a method of stimulating an immune response in a patient having an adenocarcinoma. For example, the invention provides a method of stimulating an immune response in a patient having colorectal cancer. The method can include the step of administering to the patient one or more allogeneic tumor cells, wherein the allogeneic cell stimulates an immune response to an autologous tumor cell in the patient. The administration of allogeneic tumor cells are advantageous for stimulating an immune response against a tumor in a patient without the need for isolating cells from the patient to generate such a tumor vaccine.

The invention additionally provides a method of stimulating an immune response in a patient having an adenocarcinoma, including a patient having colorectal cancer. The method includes the step of administering to the patient one or more allogeneic tumor cells, wherein the allogeneic cells stimulate a cytotoxic T lymphocyte (CTL) response to autologous tumor cells in the patient (see Example III).

The number of different allogeneic tumor cells to be administered can be varied depending on the particular needs of the vaccine. For example, a CTL response can be stimulated by one or more allogeneic tumor cells, two or more, three or more, four or more or five or more, six or more, seven or more, eight or more, nine or more, or even ten or more allogeneic tumor cells, if desired. The number of different allogeneic tumor cells to be administered can be readily determined by one skilled in the art by administering a variable number of cell lines and determining if an immune response is stimulated or an immune response is enhanced. Exemplary allogeneic tumor cells useful in the invention include SW620, COLO 205, and SW403, as well as others disclosed herein.

The invention provides a method of stimulating an immune response in a patient having an adenocarcinoma, whereby a CTL response to autologous non-tumor cells is minimized. For example, an invention method can be used to stimulate an immune response in a colorectal cancer patient. The methods of the invention are advantageous in that the allogeneic vaccine stimulates a CTL response against autologous tumor cells of the patient while minimizing a CTL response to non-tumor cells (see Example III). In particular, the invention allogeneic vaccine resulted in a minimal CTL response to peripheral blood mononuclear cells (PBMC). As used herein, a “minimized” CTL response, when used in reference to autologous non-tumor cells, refers to a CTL response against autologous non-tumor cells that is undetectable or has little or no adverse effect on the patient.

The methods of the invention are directed to treating an individual having an adenocarcinoma, including a patient having colorectal cancer. As such, the allogeneic tumor cells useful in the invention are generally adenocarcinoma cells since such cells express a variety of adenocarcinoma antigens. For example, the allogeneic tumor cells can be colorectal cancer cells having shared antigens with colon carcinoma antigens (see Example I). Allogeneic tumor cells useful in methods of the invention include the colorectal cancer cell lines SW620, COLO 205, and SW403, which have been characterized with respect to tumor associated antigens (Example I), as well as others disclosed herein.

Colon carcinoma, which is one of the most common forms of cancer, is an ideal candidate for the development of adjuvant immunotherapeutic approaches. While most patients with colon cancer are treated by tumor resection and do not exhibit clinically detectable disease immediately following surgery, many eventually relapse with disease in the liver or abdomen due to the presence of undetectable, disseminated microscopic metastases. The relative chemotherapy resistance of these recurrent colon cancer metastases further emphasizes the need for new treatment modalities, such as adjuvant immunotherapy.

Recent advances in understanding the biology of the immune system, such as the identification of immunostimulatory genes, combined with improvements in the ability to modify gene expression, have fostered a new era of tumor immunotherapy (Rosenberg et al., Ann. Intern. Med. 108:853 (1988)); Borden and Sondel, Cancer 65:800 (1990)). Vaccinations with tumor cells genetically engineered to express immunostimulatory cytokines have resulted in significant anti-tumor immune responses in several animal tumor models (Fakhrai et al., Hum. Gene Ther. 6:591 (1995); Fearon et al., Cell 60:387 (1990); Gansbacher et al., J. Exp. Med. 172:1217 (1990); Tepper et al., Cell 57:503 (1989)). The results of a Phase I clinical trial in colorectal carcinoma patients consisting of immunizations with a mixture of irradiated, autologous tumor cells and autologous fibroblasts genetically modified to express the gene for IL-2 has been previously described (Sobol et al., Hum. Gene Ther. 6:195 (1995); Sobol et al., Clin. Cancer Res. 5:2359 (1999)). The results of that study suggested that patients with colorectal cancer have low frequencies of anti-tumor cytotoxic T cell (CTL) precursors, which can potentially be increased by this form of therapeutic vaccination.

Immuno-gene therapy would be more practical if allogeneic cells could be employed for immunizations, thus obviating the need to establish and genetically modify primary fibroblast and colon tumor cultures for each patient. The rationale for the use of allogeneic tumor cells is predicated upon the expression of shared tumor associated antigens (TAA) expressed by both the tumor cells used for immunization and the patients' tumor cells (Darrow et al., J. Immunol. 142:3329 (1989)). In colon carcinoma, clonal CTL reactivity has been used to define a number of shared TAAs (Finn, Curr. Opin. Immunol. 5:701 (1993); Tsang et al., J. Natl. Cancer Inst. 87:982 (1995); Ras et al., Hum. Immunol. 53:81 (1997)). Further studies have indicated that HLA-A2 plays a major role in TAA presentation that mediates MHC-restricted tumor destruction by cytolytic T cells (CTL) (Crowley N J et al., Cancer Res. 50:492 (1990); Crowley et al., J. Immunol. 146:1692 (1991); Pandolfi et al., Cancer Res. 51:3164 (1991)). In addition, HLA-A2 is the most common MHC Class I allele, being expressed by approximately 50% of the North American population.

As disclosed herein, an allogeneic colon cancer cell line vaccine genetically modified to express the co-stimulatory molecule CD80 has been developed and characterized. The tumor cell lines selected for inclusion in the vaccine were chosen on the basis of their expression of HLA-A2, low levels of secreted immunosuppressive factors, the expression of a spectrum of TAAs representative of colon carcinomas, and their ability to induce cross-reactive CTL responses in vitro. The results disclosed herein further demonstrate that vaccination of colon cancer patients with these tumor cell lines, combined with IL-2 secreting fibroblasts, induces CTLs reactive with the patient's autologous tumor.

In addition to patients having colorectal cancer, the principles of an allogeneic tumor cell vaccine can similarly be applied to other types of cancers such as melanoma, breast, prostate and the like. The methods of the invention are particularly useful for treatment of adenocarcinomas, including colorectal, breast, prostate and lung. For each type of cancer to be treated, the vaccine can contain allogeneic tumor cells expressing antigens common to the type of cancer to be treated. In addition, a vaccine can contain allogeneic tumor cells of a different tumor type than that of the patient being treated. For example, a vaccine containing allogeneic colon carcinoma cells, such as those disclosed herein, can be used in a vaccine for stimulating an immune response in a patient having an adenocarinoma, for example, of breast, lung, prostate, and the like. Such a vaccine is useful because the allogeneic tumor cells share common antigens in different types of tumors. For example, breast and lung adenocarcinomas, as well as colon carcinoma, express CEA, as described herein.

In methods of the invention, the allogeneic tumor cell can be genetically modified to express CD80 (B7.1). Expression of CD80 has been shown to contribute to efficacious anti-tumor immunity in animal tumor models (Baskar et al., J. Exp. Med. 181:619-629 (1995)). For example, a cell can be modified to express a CD80 molecule having the nucleotide (SEQ ID NO:13) and amino acid (SEQ ID NO:14) (GenBank accession No. NM005191; Freeman et al., J. Immunol. 143:2714-2722 (1989); Selvakumar et al., Immunogenetics 36:175-181 (1992); Freeman et al., Blood 79:489-494 (1992)). In addition, a CD80 having substantially the same sequence as SEQ ID NOS:13 or 14 can be used to modify a cell line to express CD80. As used herein, the term “substantially the same sequence” refers to an amino acid sequence or nucleotide sequence encoding an amino acid sequence that retains comparable functional and biological activity characteristic of CD80/B7.1. As disclosed herein, the allogeneic tumor cell lines SW620 and COLO 205 were genetically modified to express CD80 (B7.1). Thus, allogeneic tumor cells genetically modified to express CD80 can be used to further enhance the efficacy of the allogeneic tumor cell vaccine of the invention.

In addition to a vaccine containing allogeneic tumor cells alone, the invention also provides methods in which the allogeneic tumor cells are administered with a cytokine adjuvant. The allogeneic tumor cell vaccine can include administering a cytokine such as IL-2, GM-CSF, or others, as described above. Furthermore, the cytokine adjuvant can be administered in the form of an allogeneic cell such as a fibroblast or tumor cell genetically modified to secrete a cytokine such as IL-2, GM-CSF, or other immunostimulatory cytokines (see Example II and III).

The amount of cytokine to administer can be readily determined by one skilled in the art by administering various amounts of cytokine and determining whether an immune response is enhanced, preferably without onset of serious or life-threatening side effects. The cells can be administered in various amounts to provide a desired dose of cytokine. Generally, a cytokine is administered in a dose of at least about 50 units, about 100 units, about 200 units, about 300 units, about 400 units, about 500 units, about 600 units, about 700 units, about 800 units, about 900 units, about 1000 units, about 2000 units, about 3000 units, about 4000 units, about 5000 units, or higher if such a dose enhances an immune response without causing serious or life threatening side effects for the patient. As disclosed herein, the allogeneic fibroblast cell line KMST-6 was genetically modified to secrete IL-2 and administered in various amounts to give a dose range from 0 to 4000 units of IL-2 (Example III).

Cytokine gene transfer has resulted in significant anti-tumor immune responses in several animal tumor models (Fakhrai et al., Human Gene Therapy, 6:591-601 (1995); Shawler et al., Oncology Reports, 4:135-138 (1997).; Voelker et al., Int. J. Cancer, 70:269-277 (1997)). In these studies, the transfer of cytokine genes into tumor cells reduced or abrogated the tumorigenicity of the cells after implantation into syngeneic hosts. Anti-tumor immunity in a model of colorectal carcinoma was successfully induced by immunization with a mixture of irradiated tumor cells and IL-2 transduced fibroblasts. Immunization with a mixture of irradiated tumor cells and IL-2 transduced cells induced systemic anti-tumor immunity capable of rejecting a subsequent live tumor cell challenge. Repeated immunizations with a mixture of irradiated tumor cells and IL-2 transduced fibroblasts abolished established, visible tumors in a subset of the treated animals.

Encouraging results have been obtained with an autologous tumor vaccine as an adjuvant therapy following tumor resection. Immunization with autologous tumor preparations and the adjuvant BCG resulted in a significant increase in disease free and total survival (Hoover et al., J. Clin. Oncol., 11:390 (1993)). These findings, combined with the demonstration of enhanced anti-tumor immunity following tumor immunizations with cells genetically modified to express IL-2 in several animal tumor systems, provide support for using IL-2 gene transfer for tumor therapy.

Colorectal carcinoma is one of the most common cancers in industrialized nations. Previous studies in human subjects have indicated that colorectal carcinomas are sensitive to therapy with immunomodulators (Hoover et al., J. Clin Oncol., 11:390-399 (1993); Herlyn et al., International REviews of Immunology, 7:2445-257 (1991); Herlyn et al., J. Immunotherapy, 15:303-311 (1994)). The invention is directed to developing active immunotherapy for adenocarcinoma, including colon carcinoma, which is inadequately treated by conventional methods. As disclosed herein, the effects of two different types of genetic manipulations to enhance the efficacy of therapeutic tumor vaccines were examined. The effect of expression of the immunostimulatory cytokine IL-2 by genetically transfecting the gene for its expression into immortalized fibroblasts and co-expression in two of three colon tumor cell lines of the co-stimulatory molecule B7.1 (CD80) were examined. Preliminary results indicated that a KMST fibroblast line engineered to secrete IL-2 mixed with autologous tumor cells demonstrated no significant adverse reactions and resulted in generation of anti-tumor immune responses (Veelken et al., Int. J. Cancer, 70:267-277 (1997)).

Immunization with autologous tumor preparations and the adjuvant BCG resulted in a significant increase in disease free and total survival (Hoover et al., J. Clin Oncol., 11:390-399 (1993)). Additional studies have suggested therapeutic efficacy of passive and active immunotherapies directed against the TAA recognized by the monoclonal antibodies 17-1A and GA733 (Herlyn et al., International REviews of Immunology, 7:2445-257 (1991); Herlyn et al., J. Immunotherapy, 15:303-311 (1994)).

Recent studies of the biology of the immune system have led to the identification of numerous cytokines which modulate immune responses (Kelso, Curr. Opin. Immunol., 2:215-225 (1989); Borden and Sondel, Cancer, 65:800-814 (1990)). These agents mediate many of the immune responses involved in anti-tumor immunity. Several of these cytokines have been produced by recombinant DNA methodology and evaluated for their anti-tumor effects. In experimental clinical trials, the administration of cytokines and related immunomodulators has resulted in objective tumor responses in some patients with various types of neoplasms (Borden and Sondel, Cancer, 65:800-814 (1990); Rosenberg et al., Ann Intern. Med., 108:853-864 (1988); Lotze et al., JAMA 256:3117-3124 (1986)).

Interleukin-2 (IL-2) is an important cytokine in the generation of anti-tumor immunity (Rosenberg et al., Ann Intern. Med., 108:853-864 (1988)). In response to tumor antigens, the helper T-cell subset of lymphocytes secretes small quantities of IL-2. This IL-2 acts locally at the site of tumor antigen presentation to activate cytotoxic T-cells and natural killer cells which mediate systemic tumor cell destruction. Intravenous, intralymphatic or intralesional administration of IL-2 has resulted in clinically significant responses in several types of cancer (Rosenberg et al., Ann Intern. Med., 108:853-864 (1988); Lotze et al., JAMA 256:3117-3124 (1986); Pizza et al., Cytokine Research, 7:45-48 (1988); Sarna et al., J. Biol. Response Modifiers, 9:81-86 (1990); Gandolfi et al., Hepato-Gastroenterology, 36:352-356 (1989)). However, severe toxicities (hypotension and edema) limit the dose and efficacy of intravenous and intralymphatic IL-2 administration (Lotze et al., JAMA 256:3117-3124 (1986); Pizza et al., Cytokine Research, 7:45-48 (1988); Sarna et al., J. Biol. Response Modifiers, 9:81-86 (1990)). The toxicity of systemically administered cytokines is not surprising since these agents mediate local cellular interactions and are normally secreted in quantities too small to have systemic effects.

To circumvent the toxicity of systemic IL-2 administration, several investigators have examined intralesional injection of IL-2 (Gandolfi et al., Hepato-Gastroenteroloqy, 36:352-356 (1989); Bubenik et al., Immunology Letters, 19:279-282 (1988)). This approach greatly reduced the toxicity associated with systemic IL-2 administration. However, multiple intralesional injections were required to optimize therapeutic efficacy (Gandolfi et al., Hepato-Gastroenterology, 36:352-356 (1989); Bubenik et al., Immunology Letters, 19:279-282 (1988)). Multiple injections are impractical for many patients, particularly when tumor sites are not accessible for direct injection without potential significant morbidity. Importantly, the ultimate application for vaccines is in patients who have no evidence of tumor following surgery.

Cytokine gene transfer has resulted in significant anti-tumor immune responses in several animal tumor models (Fearon et al., Cell, 60:387-403 (1990); Gansbacher et al., J. Exp Med., 172:1217-1223 (1990); Watanabe et al., Proc. Natl. Acad. Sci. USA, 86:9456-9460 (1989); Tepper et al., Cell, 57:503-512 (1989)). In these studies, the transfer of cytokine genes into tumor cells has reduced or abrogated the tumorigenicity of the cells after implantation into syngeneic hosts. The transfer of genes for IL-2 (Fearon et al., Cell, 60:387-403 (1990); Gansbacher et al., J. Exp Med., 172:1217-1223 (1990)); gamma interferon (IFN) (Watanabe et al., Proc. Natl. Acad. Sci. USA, 86:9456-9460 (1989)); and IL-4 (Tepper et al., Cell, 57:503-512 (1989)) significantly reduced or eliminated the growth of several different histological types of murine tumors. In studies employing IL-2 gene transfer, the treated animals also developed systemic anti-tumor immunity and were protected against subsequent tumor challenges with the unmodified parental tumor (Fearon et al., Cell, 60:387-403 (1990); Gansbacher et al., J. Exp Med., 172:1217-1223 (1990)). Similar inhibition of tumor growth and protective immunity were also demonstrated when immunizations were performed with a mixture of unmodified parental tumor cells and genetically modified tumor cells engineered to express the IL-2 gene. No toxicity associated with expression of the cytokine transgenes was reported in these animal tumor studies (Fearon et al., Cell, 60:387-403 (1990); Gansbacher et al., J. Exp Med., 172:1217-1223 (1990); Watanabe et al., Proc. Natl. Acad. Sci. USA, 86:9456-9460 (1989); Tepper et al., Cell, 57:503-512 (1989)).

Unfortunately, many types of tumors are difficult to establish in culture, and cytokine gene therapies requiring the transduction of autologous tumor cells can be impractical for many cancer patients. Furthermore, current vector systems are limited in the number of genes that can be transferred into tumor cells. However, primary human fibroblasts obtained from skin biopsies or established allogeneic fibroblast cell lines are readily cultured in vitro and genetically modified to express and secrete cytokines (Fakhrai et al., Human Gene Therapy, 6:591-601 (1995); Sobol et al., Gene Therapy, 2:164-167 (1995); Kim et al., Cancer Research, 54:2531-2535 (1994); Kim et al., International J. Cancer, 51:283-289 (1992); Tahara et al., Cancer Research, 54:182-189 (1994)). The genetically modified, irradiated fibroblasts are then mixed with irradiated autologous or allogeneic tumor cells and employed in immunizations to induce systemic anti-tumor immunity. Application of genetically modified fibroblasts in therapeutic vaccines facilitates titration of single or multiple cytokine doses independent of tumor cell doses and permits other forms of genetic manipulation to be performed on the tumor cell component of the vaccines to further enhance its immunogenicity. These considerations provide the rationale for examining the use of fibroblasts genetically modified to secrete cytokines as a means of enhancing anti-tumor immune responses, as disclosed herein.

Several groups have demonstrated the efficacy of active tumor immunotherapy with cytokine-transduced syngeneic or allogeneic fibroblasts in tumor animal models (Fakhrai et al., Human Gene Therapy, 6:591-601 (1995); Sobol et al., Gene Therapy, 2:164-167 (1995); Kim et al., Cancer Research, 54:2531-2535 (1994); Kim et al., International J. Cancer, 51:283-289 (1992); Tahara et al., Cancer Research, 54:182-189 (1994)). In studies employing a murine colon tumor model, immunizations with a mixture of irradiated tumor cells and irradiated IL-2 transduced fibroblasts generated systemic anti-tumor immunity capable of rejecting a subsequent tumor challenge and eradicating established tumors (Fakhrai et al., Human Gene Therapy, 6:591-601 (1995)). In subsequent work in this same model, it was also shown that allogeneic fibroblasts engineered to secrete IL-2 were equally effective in eliciting antitumor responses when compared to syngeneic fibroblasts (Shawler et al., Oncology Reports, 4:135-138 (1994)). Similar results were observed in a murine melanoma model following immunizations with tumor cells and allogeneic fibroblasts genetically modified to express IL-12 (Tahara et al., Cancer Research, 54:182-189 (1994)).

In related animal studies, systemic anti-tumor immunity was induced following immunization with IL-2 secreting fibroblasts that were also transfected with tumor DNA (Kim et al., Cancer Research, 54:2531-2535 (1994); Kim et al., International J. Cancer, 51:283-289 (1992)). In these studies, the results of immunizations with allogeneic and syngeneic fibroblasts transfected with tumor DNA were compared with and without concomitant IL-2 gene transfer. It was found that different types of effector cells were induced by immunizations with IL-2 transduced autologous versus allogeneic fibroblasts and that combined IL-2 gene transfer and allogeneic stimulation had synergistic effects with enhanced survival compared to immunization with either approach alone (Kim et al., Cancer Research, 54:2531-2535 (1994). Kim et al., International J. Cancer, 51:283-289 (1992)).

As disclosed herein, tumor vaccines containing fibroblasts genetically modified to secrete cytokines were effective as a means of enhancing anti-tumor immune responses. A 10 patient study in recurrent colorectal cancer comprised of injection of autologous tumor cells mixed with autologous IL-2 secreting fibroblasts showed the vaccine to be safe and able to elicit immune responses against the tumor. As disclosed herein, the methods of the invention are advantageous in that the use of allogeneic cell lines avoids the need of individualized therapies for such patients.

Recently, clinical work with allogeneic fibroblasts secreting IL-2 in combination with autologous tumor cells was shown to be safe and capable of producing tumor specific immune responses in patients with advanced cancers. (Veelken et al., Int. J. Cancer, 70:267-277 (1997)). The immortalized KMST-6.14 fibroblast cell line was used in the clinical trials disclosed herein for several reasons. First, the line grows very well and was successfully transfected with the IL-2 gene such that IL-2 was efficiently made and secreted. Secondly, the line has been extensively studied pre-clinically and documented to be non-tumorogenic. Finally, the line has been clinically utilized with no apparent signs of significant toxicity in early clinical trials in Germany (Veelken et al., Int. J. Cancer, 70:267-277 (1997)).

It has become increasingly clear that antigen recognition alone is not sufficient for optimal activation of T cell effector functions. “Second signals” such as co-ligation of auxiliary molecules are also critical for generating T cell mediated immunity (Mondino et al., J. Leukocyte Biol., 55:805-815 (1994); June et al., Immunol. Today, 11:211-216 (1990)). Furthermore, antigen recognition in the absence of these second signals can lead to tolerance or “anergy” (Mondino et al., J. Leukocyte Biol., 55:805-815 (1994); June et al., Immunol. Today, 11:211-216 (1990)). Two co-stimulatory molecules in particular, B7.1 (CD80) and B7.2 (CD86), the ligands for CD28 and CTLA-4, have recently received a great deal of attention as potent co-stimulators for T cell function. In humans, B7.1 is expressed on professional antigen presenting cells (APCs), including dendritic cells, and is induced on activated B cells, T cells, NK cells and macrophages (Azuma et al., J. Exp. Med., 177:845-850 (1993); Freeman et al., J. Immunol., 143:2714-2722 (1989)). Northern analysis for mRNA expression of B7 revealed that most carcinomas, leukemias of B cell origin (including non-T cell acute lymphoblastic leukemia (ALL)), prolymphocytic leukemia, hairy cell leukemia and chronic lymphocytic leukemia were B7.1 negative while some non-Hodgkin's lymphomas were positive (Freeman et al., J. Immunol., 143:2714-2722 (1989)). These results suggested that lack of B7.1 expression by many tumors can contribute to their poor immunogenicity. Similarly, the majority of colon cancer cell lines, including those selected for the clinical trials disclosed herein, are B7.1 negative.

In previous studies, transfection of the B7.1 gene into murine melanoma and sarcoma models caused the transfected tumors to be rejected in vivo (Townsend et al., Science, 259:368-370 (1993); Sivasubramanian et al., Proc. Natl. Acad. Sci. USA, 90:5687-5689 (1993)). In both cases, once immunity was induced, the animals were protected from challenge with the unmodified tumor. Since this immunity was dependent on the presence of cytolytic T cells, an important conclusion is that the presence of B7 on the tumor is critical for T cell induction but not for effector cell function. These studies also suggested that the absence of appropriate co-stimulatory molecules on tumors could be a critical factor allowing escape from immune attack despite the expression of potentially strong tumor associated antigens. In more recent studies, combined cytokine, including IL-2, and B7.1 gene transfer demonstrated synergistic effects in generating efficacious anti-tumor immunity in animal tumor models (Hollingsworth et al., Cancer Gene Therapy, 2:240 (1995)). The clinical trial studies in patients with colorectal carcinoma using such a combined approach was effective at stimulating a CTL response to autologous tumor cells, as disclosed herein.

Immuno-gene therapy is more practical if allogeneic cells are employed for immunizations, which obviates the need to establish primary fibroblast and colon tumor cultures for each patient. The rationale for the use of allogeneic tumor cells is predicated upon the expression of shared tumor associated antigens (TAA) between the tumor used for immunization and the patients' tumors. Established lines can be carefully characterized and selected for optimal characteristics and can also be genetically modified to express additional gene products, as disclosed herein. Using the methods of the invention, the content of diverse antigens is further increased through using three different cell lines in the vaccine rather than just one. Several studies have indicated that HLA-A1, HLA-A2 and HLA-A3 haplotypes play a major role in shared TAA presentation, which can mediate MHC-restricted tumor destruction by cytolytic T cells (CTL) (Crowley et al., Cancer Research, 50:492 (1990); Crowley et al., J. Immunol., 146:1692-1699 (1991); Pandolfini et al., Cancer Res., 51:3164-3170 (1991); Chen et al., Cancer Immunol Immunotherapy, 38:385-393 (1994)). In addition, the HLA-A1, HLA-A2 and HLA-A3 haplotypes are relatively common, being expressed by approximately 25%, 50% and 20% of the North American population, respectively.

As disclosed herein, a vaccine containing 3 colon tumor cell lines that are HLA-A2 positive was used to effect a CTL response in HLA-A2 positive patients. Several shared tumor TAAs defined by CTLs have been described in colon carcinomas (Finn et al., Current Opinion in Immunology, 5:701-708 (1993); De Plaen et al., Immunogenetics, 40:360-369 (1994)). The protein components of tumor mucin (MUC-1) and the melanoma antigen (MAGE) gene family are TAAs expressed by many colon carcinomas and other adenocarcinomas (Finn et al., Current Opinion in Immunology, 5:701-708 (1993); De Plaen et al., Immunogenetics, 40:360-369 (1994)). Additional TAAs expressed by the majority of colon carcinomas include CEA and the glycoprotein recognized by the monoclonal antibodies CO-17-1A and GA733 (Herlyn et al., International REviews of Immunology, 7:2445-257 (1991); Herlyn et al., J. Immunotherapy, 15:303-311 (1994)).

For immuno-gene therapy, the use of allogeneic cells for immunizations obviates the need to establish and genetically modify primary fibroblast and adenocarcinoma such as colon tumor cultures for each patient. The rationale for the use of allogeneic tumor cells is predicated upon the expression of shared tumor associated antigens (TAA) expressed by both the tumor cells used for immunization and the patients' tumor cells (Darrow et al., J. Immunol., 142:3329-3335 (1989)). In colon carcinoma, clonal CTL reactivity has been used to define a number of shared TAAs (Finn, Curr. Op. Immunol., 5:701-708 (1993); Tsang et al., J. Natl. Cancer Inst., 87:982-990 (1995); Ras et al., Hum. Immunol., 53:81-89 (1997)). These studies have indicated that the HLA-A2 haplotype plays a major role in TAA presentation that mediates MHC-restricted tumor destruction by cytolytic T cells (CTL) (Crowley et al., Cancer Res., 50:492-498 (1990); Crowley et al., J. Immunol., 146:1692-1699 (1991); Pandolfi et al., Cancer Res., 51:3164-3170 (1991)). In addition, HLA-A2 is the most common MHC class I haplotype, being expressed by approximately 50% of the North American population.

The generation of an allogeneic fibroblast cell line genetically engineered to express IL-2 is disclosed herein (see Examples I and II). A vaccine containing allogeneic colon cancer cell lines genetically modified to express the co-stimulatory molecule CD80 was developed and characterized (Example I). The cell lines selected for inclusion in the vaccine were chosen on the basis of their expression of HLA-A2, low levels of secreted immunosuppressive factors and the expression of a spectrum of TAAs representative of colon carcinomas.

As disclosed herein, a practical allogeneic tumor cell vaccine was developed for the immuno-gene therapy of colon cancer based on the immunologic profiles of established colon carcinoma cell lines compared to fresh colon carcinoma cultures initiated from biopsy material. The vaccine consisted of three established cell lines, SW620, COLO 205, and SW403, that are HLA-A2 positive; do not secrete high levels of the immunosuppressive factors TGF-β1 and -β2, IL-10, or prostaglandins; and collectively express a spectrum of putative tumor associated antigens (TAAs) representative of colon carcinomas: carcinoembryonic antigen (CEA), Ep-CAM, p53, HER-2/neu, MUC1 and MAGE 2, 3, 4, 6, and 12. These cell lines all express HLA-A2.1, the most common HLA-A allele of the major histocompatibility complex (MHC), which plays a major role in MHC-restricted tumor destruction by cytolytic T-lymphocytes (CTLs).

To demonstrate that these lines could present TAAs in a manner recognized by immune effector cells, it was shown that SW620 cells, which overexpresses p53, could be lysed by HLA-A2.1-restricted CTL that recognize a p53 epitope. Two of the three lines (COLO 205 and SW620) were genetically modified to express the co-stimulatory molecule CD80 (B7.1), which increased the ability of these cells to stimulate CTL in vitro. To determine if these lines could induce CTL that recognize shared TAA, CTL clones derived from normal HLA-A2 positive peripheral blood mononuclear cells stimulated with the CD80-expressing lines were tested for their ability to lyse a panel of target cells. Clones from these cultures lysed the stimulator cell and an HLA-A2 positive colon cancer cell line, but did not lyse an isogenic fibroblast line. These clones also failed to lyse an HLA-A2 negative colon cancer cell line, suggesting that they recognized shared HLA 2.1 restricted TAA. Clones derived from colon carcinoma patients immunized with an allogeneic vaccine containing these lines demonstrated killing of autologous tumor cells, the vaccine cell lines, and other HLA-A2 positive colon cancer cell lines, but not fibroblasts isogenic to certain of these target cell lines. These results disclosed herein demonstrate that these colon carcinoma cell lines express shared TAAs that can induce CTLs that recognize and lyse other colon carcinoma cells and support the continued clinical evaluation of the CD80 gene modified allogeneic colon cell/cytokine secreting fibroblast carcinoma vaccine.

An effective allogeneic vaccine induces cytotoxic T lymphocytes (CTLs) to shared TAAs. Stimulation of normal HLA-A2 positive peripheral blood mononuclear cells with SW620 induced CTL that lysed SW620 and the HLA-A2 positive fresh colon cells GT53T but did not lyse GT53F, a fibroblast line isogenic to GT53T, or HT-29, an HLA-A2 negative colon cell line (see Example I). Genetic modification of tumor cell lines to express the co-stimulatory molecule CD80 (B7.1) increased their ability to stimulate CTL in vitro. The results disclosed herein demonstrate that primary and established colon carcinoma cell lines have similar immunologic characteristics and express TAAs that CTL can recognize as shared TAAs.

The ideal tumor cell vaccine expresses appropriate MHC and co-stimulatory molecule, secrete only low levels of immunosuppressive factors, and present a spectrum of shared TAAs representative of patients' tumors. Through the process of screening established cell lines and genetic engineering, a tumor cell vaccine for colorectal cancer has been developed and characterized that approximates such an ideal vaccine. From a group of 18 established colon lines, three cell lines were identified that met the criteria for forming an allogeneic vaccine: SW620, COLO 205, and SW403. These cell lines were HLA-A2 positive, did not secrete high levels of immunosuppressive factors and expressed a profile of putative tumor antigens representative of colon carcinomas. The SW620 and COLO 205 cell lines were genetically modified to express the co-stimulatory molecule CD80 to enhance their immunogenicity (see Example I).

To ensure that the colon cancer vaccine was representative of patients' tumors, the immunologic profiles of the established colon carcinoma cell lines used in the vaccine were compared to fresh colon carcinoma cell cultures derived from patients' primary and metastatic tumors. The results disclosed herein demonstrate that the immunologic characteristics of the two sets of colon tumor cells are similar. All of the cell lines tested expressed MHC Class I, but not MHC Class II antigens. Cell lines from both groups secreted a similar range of immunosuppressive factors and expressed a similar variety of shared TAAs. These results demonstrate that a carefully selected pool of allogeneic colon carcinoma cell lines provides an immunologic profile representative of many patients' tumors.

TGF-β1 and TGF-β2 are potent immunosuppressive factors (Sporn et al., Science, 233:532-534 (1986); Massague, Cell, 49:437-438 (1987); Inge et al., Cancer Res., 52:1386-1392 (1992)), frequently secreted by tumor cells of various histologies (MacCallum et al., Br. J. Cancer, 69:1006-1009 (1994); Troung et al., Hum. Pathol., 24:4-9 (1993)). As disclosed herein, approximately three-quarters of both the fresh colon carcinoma cultures and the established colon carcinoma cell lines created TGF-β1 (Example I). Anzano et al. reported that 77% of established colon lines secreted TGF-β1 in a range of 125 to 8000 pg/10⁶ cells/24 hr (Anzano et al., Cancer Res., 49:2898-2904 (1989)). In contrast to TGF-β1 secretion, only one-quarter of the fresh and established colon carcinoma cell lines evaluated secreted TGF-β2 (Example I). Of the three colon lines chosen for the vaccine, COLO 205 and SW620 secrete low levels of TGF-β1, SW403 does not secrete TGF-β1, and none of the three lines secrete TGF-β2. Animal tumor models previously demonstrated that partial inhibition of TGF-β secretion by antisense molecules significantly improves the efficacy of tumor cell vaccination, thus indicating the importance of minimizing the amount of immunosuppressive factors secreted by tumor cell vaccines (Fakhrai et al., Proc. Natl. Acad. Sci. (USA), 93:2909-2914 (1996); Dorigo et al., Gynecol. Oncol. 71:204-210 (1998)).

Colon carcinomas are known to express a variety of shared putative TAAs. As disclosed herein, both fresh colon carcinoma cell cultures and established colon carcinoma cell lines express a number of previously characterized TAAs including CEA (Muraro et al., Cancer Res., 45:5769-5780 (1985); Han and Nair, Cancer, 76:195-200 (1995)), MUC-1 (Hanski et al., Cancer Res., 53:4082-4088 (1993)), EPCAM (Herlyn et al., Proc. Natl. Acad. Sci. (USA), 76:1438-1442 (1979); Litvinov et al., J. Cell Biol., 125:437-446 (1994)), HER-2/neu (Conry et al., Clin. Cancer Res., 5:2330-2337 (1999)), and the MAGE family (De Plaen et al., Immunogenetics, 40:360-369 (1994)), as well as p53 mutations (Nigro et al., Nature, 342:705-708 (1989); Pricolo et al., Arch. Surg., 132:371-374 (1997)), all of which have been associated with HLA-A2-mediated CTLs (see Example I). CEA is perhaps the best characterized colon carcinoma-associated antigen. It is expressed in 80% of colon cancers (Muraro et al., Cancer Res., 45:5769-5780 (1985); Han and Nair, Cancer, 76:195-200 (1995)), has been demonstrated to be the target of both humoral and cellular immune responses (Conry et al., Clin. Cancer Res., 5:2330-2337 (1999); Fagerberg et al., J. Immunother., 19:461 (1996)); and contains HLA-A2 binding epitopes (Ras et al., Hum. Immunol., 53:81-89 (1997); Muraro et al., Cancer Res., 45:5769-5780 (1985); Zaremba et al., Cancer Res., 57:4570-4577 (1997)). Both the fresh and established colon lines described herein demonstrated CEA expression similar to that observed previously (Han and Nair, Cancer, 76:195-200 (1995)).

Ep-CAM, MUC-1, and HER-2/neu were also expressed by most of the fresh and established tumor cell lines described herein (Example I). Ep-CAM is a colon carcinoma-associated cell surface antigen (Litvinov et al., J. Cell Biol., 125:437-446 (1994)) that has been demonstrated to be an important target for both humoral (Riethmuller et al., J. Clin. Oncol., 16:1788-1794 (1998)), and cellular immunity (Ras et al., Hum. Immunol., 53:81-89 (1997)). MUC-1 is an unusual antigen that can mediate MHC restricted and MHC unrestricted cytotoxicity, presumably through the cross-linking of T cell receptors by repetitive amino acid sequences (Finn, Curr. Op. Immunol., 5:701-708 (1993)). HER-2/neu is a well-characterized TAA that can function as an antigen for HLA-A2 directed CTL (Lustgarten et al., Hum. Immunol., 52:109-118 (1997)).

The tumor suppressor gene p53 is abnormally expressed in half of colon carcinomas (Nigro et al., Nature, 342:705-708 (1989); Pricolo et al., Arch. Surg., 132:371-374 (1997)). Importantly, an HLA-A2-binding p53 epitope corresponding to a wild type amino acid sequence has recently been identified (Rdpke et al., Proc. Natl. Acad. Sci. (USA), 93:14704-14707 (1996)). Human CTL can target this shared epitope in tumor cells that overexpress p53 (Gnjatic et al., (Gnjatic et al., J. Immunol. 160:328-333 (1998)).

As disclosed herein, the MAGE gene family is frequently expressed in colon carcinomas (Example I). MAGE-1 was initially characterized as a tumor-associated antigen in melanoma recognized by CTLs (van der Bruggen et al., Science, 254:1643-1647 (1991)). This initial observation has been extended to include a family of MAGE proteins (De Plaen et al., Immunogenetics, 40:360-369 (1994)), expressed by tumors of varying histological types (Brasseur et al., Int. J. Cancer, 52:839-841 (1992); Shichijo et al., Int. J. Cancer, 64:158-165 (1995)). MAGE gene products have been demonstrated to induce potent HLA-A2-restricted CTL (van der Bruggen et al., Eur. J. Immunol., 12:3038-3043 (1994); Celis et al., Mol. Immunol., 18:1423-1430 (1994)). Hence, the results disclosed herein that multiple MAGE genes are expressed by both fresh and established colon cell lines is important in the context of shared tumor-associated antigens for vaccine development.

Based on the results disclosed herein, the cell lines SW620, COLO 205, and SW403 were chosen for an allogeneic cell vaccine for colon cancer. These cells share several TAAs commonly expressed by colon carcinomas: CEA, MUC-1, Ep-CAM, HER-2/neu, p53, and MAGE (Ho et al., Mol. Carcinog., 16:20-31 (1996); Rodriguez et al., Proc. Natl. Acad. Sci. USA, 87:7555-7559 (1990); Cottu et al., Oncogene, 13:2727-2730 (1996)). For an allogeneic vaccine to be effective, it must be able to induce CTLs capable of lysing autologous tumor cells. As disclosed herein stimulation of normal HLA-A2 positive PBMC with irradiated SW620 cells induced CTL that lysed the established colon lines SW620 as well as the fresh colon culture GT53T (Example I). These in vitro studies demonstrate that HLA-A2 positive allogeneic tumor cells can induce cross-reactive CTL that recognize shared tumor-associated antigens (Example I).

For an allogeneic vaccine to be effective, it must present TAAs in a manner recognized by the immune system, and it must induce CTLs capable of lysing autologous tumor cells. As disclosed herein, both of these characteristics were found using one of the cells chosen for a vaccine, SW620 (IR806). Firstly, it was demonstrated that HLA-A2-restricted anti-p53 CTLs, which recognize the wild type amino acid sequence 264-272 of p53, could lyse SW620 cells, which overexpress a mutated form of p53 (Nigro et al., Nature 342:705 (1989); Rodrigues et al., Proc. Natl. Acad. Sci. USA 87:7555 (1990)). Secondly, it was demonstrated that stimulation of normal HLA-A2 positive PBMC with irradiated IR806 cells induced CTL that lysed the established colon lines IR806 as well as the fresh colon culture GT53T. The CTL did not lyse GT53F, a skin fibroblast line autologous to GT53T, nor did it lyse the HLA-A2 negative colon carcinoma line HT-29. These in vitro studies demonstrated that HLA-A2 positive allogeneic tumor cells can induce cross-reactive CTL presumably due to the expression of shared tumor-associated antigens.

As disclosed herein, transfection of colon cancer cells with CD80 improved their ability to stimulate colon tumor CTL. These findings support the inclusion of CD80 gene modified cells in a colon cancer cell lines vaccine. Antigen recognition alone is not sufficient for T cell activation to effector function. Second signals such as co-ligation of auxiliary molecules are also critical for generating T cell mediated immunity (Mondino and Jenkins, J. Leukocyte Biol., 55:805-815 (1994); June et al., Immunology Today, 11:211-216 (1990)). The co-stimulatory molecule CD80(B7.1), which is the ligand for CD28, is a potent co-stimulatory for T cell function. In humans, CD80 is constitutively expressed on dendritic cells and is induced on activated B cells, T cells, NK cells and macrophages (Azuma et al., J. Exp. Med., 177:845-850 (1993); Freeman et al., J. Immunol., 143:2714-2722 (1989)). Thus, costimulation by host antigen presenting cells can occur without the need for costimulatory molecules on the tumor cells. In previous studies, transfection of the CD80 gene into murine tumors induced in vitro rejection of the transfected tumors (Hsu et al., Cell Growth & Differ. 5:267-275 (1994); Friedman et al., Cancer Epidermal Biomarkers Prev., 4:549-554 (1995)). Once immunity was induced, the animals were protected from challenge with the unmodified tumor. Thus, the presence of CD80 on the tumor is likely important for T cell induction but not for effector cell function. Studies in other systems have indicated that antigen recognition in the absence of CD80 co-stimulatory signals can lead to tolerance or anergy (Mondino and Jenkins, J. Leukocyte Biol., 55:805-815 (1994); June et al., Immunology Today, 11:211-216 (1990)). The absence of appropriate co-stimulatory molecules on tumors can contribute to their escape from immune surveillance despite the expression of potentially strong TAAs. As disclosed herein, transfection of colon cancer cells with CD80 improved their ability to stimulate colon tumor CTL.

Ultimately, immunization with allogeneic tumor cell lines should provide immunity to a patient's own tumor. As disclosed herein, CTL clones were developed from the PBMC of patients who were immunized with a vaccine composed of these lines mixed with IL-2-secreting fibroblasts. These genetically modified tumor cells are capable of inducing CTLs specific for a patient's own tumor. The observation that these clones exhibited specificity for the immunizing lines and the autologous tumor suggest that these lines could induce colon carcinoma-restricted responses in HLA-A2.1 subjects.

The invention additionally provides a method of enhancing an anti-tumor immune response in a patient having an adenocarcinoma, including a patient having colorectal cancer, by administering one or more allogeneic tumor cells, wherein the administration stimulates cytotoxic T cell precursors specific for an autologous tumor in the patient. The invention further provides a method of enhancing an anti-tumor immune response in a patient having an adenocarcinoma, including a patient having colorectal cancer, by administering one or more allogeneic tumor cells, wherein the allogeneic cells stimulate cytotoxic T lymphocytes (CTL) specific for autologous tumor cells and whereby a CTL response to autologous non-tumor cells is minimized.

The invention also provides a method of enhancing an immune response in a patient having an adenocarcinoma, including a patient having colorectal cancer. The method includes the steps of administering to the patient one or more allogeneic tumor cells, wherein the allogeneic cells stimulate cytotoxic T lymophocytes (CTL) specific for autologous tumor cells; isolating a CTL clone specific for autologous tumor cells; amplifying said CTL clone in vitro; and administering the amplified CTL clone to the patient.

As disclosed herein, an allogeneic tumor cell vaccine stimulated a CTL response (Example III). CTL clones derived from post-vaccination PBMCs killed autologous tumor cells and vaccinating cell lines. Such CTL lines can be further amplified in vitro and re-administered to an individual to enhance an immune response.

It is understood that modifications which do not substantially affect the activity of the various embodiments of this invention are also provided within the definition of the invention provided herein. Accordingly, the following examples are intended to illustrate but not limit the present invention.

### EXAMPLE I

### Development and Characterization of an Allogeneic Colon Carcinoma Vaccine

This example describes development and characterization of an allogeneic colon carcinoma vaccine comprised of established cell lines.

To establish fresh colon carcinoma cell cultures, biopsy specimens were obtained from 61 colon carcinoma patients undergoing tumor resections or CT-guided biopsies for therapeutic purposes. From these, 41 proliferating cell lines were established (67%). Biopsies were placed in sterile 50 ml tubes in an excess of culture media composed of Dulbeccos Modified Minimum Essential Media (Mediatech, Inc.; Herndon, Va.) supplemented with 10% fetal bovine serum (Gemini Bioproducts; Calabasas, Calif.), and 50 Hg/ml of both gentamycin and amphotericin B (Sigma Chemical Co.; St. Louis, Mo.). For specimens obtained from the colon, the concentration of gentamycin was increased to 150 kg/ml. Non-tumor and necrotic tissue were removed from the viable tumor using number 21 scalpels. The remaining viable tumor was minced into 3-5 mm pieces and washed 3× with culture media. The minced tumor was placed in culture media supplemented with 300 U/ml collagenase and 200 U/ml DNase (Sigma Chemical Co.) and incubated overnight in a humidified 10% CO₂ atmosphere at 37° C. The tumor pieces were then washed 3× in serum-free culture media, resuspended in 5 ml of 100 mM trypsin-EDTA (Mediatech, Inc.), and incubated for 5 minutes at 37° C. The reaction was stopped by the addition of 1 ml cold fetal bovine serum and the tumor was again washed 3× in culture media.

The digested tumor was resuspended in culture media, placed in 225 cm² tissue culture flasks (Costar Inc.; Pleasanton, Calif.), and cultured in a humidified 10% CO₂ atmosphere at 37° C. After 3 days, the nonadherent cells and debris were washed from the flask and fresh culture medium was added. The cultures were maintained with replacements of media twice per week. When grown to confluency, the cells were harvested by trypsin-EDTA and seeded into new flasks. When necessary, fibroblasts were depleted from the cultures using the protocol of Dillman et al. (Dillman et al., J. Immunother., 14:65-69 (1993)).

The established colon carcinoma cell lines used, including COLO 205, SW620, and SW403, were all obtained from the American Type Culture Collection (ATCC; Manassas, Va.). The colon carcinoma cell lines SW620 and COLO 205 were genetically modified to constitutively express the costimulatory molecule CD80 (B7.1). The resulting cell lines were termed IR806 and IR804, respectively.

The cells were maintained in routine tissue culture using Dulbecco's Modified Minimum Essential Media (Mediatech; Herndon Va.) culture media supplemented with 10% fetal bovine serum (Gemini Bioproducts; Calabasas Calif.) and 50 μg/ml of both gentamycin and amphotericin B (Sigma Chemical Co.; St. Louis MO). The cells were seeded into 75 to 225 cm² tissue culture flasks and were placed in a 37° C., 10% CO₂ incubator with twice weekly changes of media until confluency. The cells were harvested by trypsin/EDTA and reseeded into fresh tissue culture flasks.

Fifteen fresh colon carcinoma cultures and 9 established colon carcinoma cell lines were assayed by immunofluorescence flow cytometry for the expression of immune accessory molecules. All cells tested were positive for MHC Class 1 and I-CAM expression and negative for MHC class II and CD80 expression. HLA-A2 (the most commonly expressed human MHC Class I antigen) was expressed by 56% of the fresh cell cultures (10 of 18) and by 43% of the established cell lines (6 of 14).

Many histological types of tumor cells, including colon carcinoma cells secrete TGF-β (Sporn et al., Science 233:532 (1986); Massague, Cell 49:437 (1987)), a potent immunosuppressive factor that has been shown to inhibit the efficacy of cancer vaccine therapy (Fakhrai et al., Proc. Natl. Acad. Sci. USA 93:2909 (1996); Dorigo et al., Gynecol. Oncol. 71:204 (1998)). TGF-β, IL-10 and prostaglandins are known to be potent immunosuppressive factors secreted by many histological types of tumor cells. The secretion of these factors was therefore examined by both fresh cultures and established colon carcinoma cell lines. To measure TGF-β, IL-10, and prostaglandin-2 secretion by cell lines, cells were plated in 6-well plates (Costar, Inc.) in culture media at concentrations of 1×10⁶, 5×10⁵, and 2.5×10⁵ cells per well. The next day, the culture media was removed, the cells were washed extensively with serum-free DMEM, and fed with 4 ml serum-free DMEM. After 24 hours, the supernatants were collected, placed into 1.5 ml polystyrene tubes that had been precoated with 0.1% bovine serum albumin to provent TGF-β adsorption by the plastic, and stored at −70° C. TGF-β was activated by the addition of 100 μl 1.0 N HCL to 100 μl supernatant for 5 minutes at room temperature followed by neutralization with 100 μl supernatant for 5 minutes at room temperature followed by neutralization with 100 μl of 1.0 N NaOH.

TGF-β1, TGF-β2, and IL-10 concentrations were determined using commercially available enzyme-linked immunosorbent assays (ELISAs) (R&D Systems; Minneapolis, Minn.). Prostaglandin-2 concentrations were determined using a commercially available ELISA (PerSeptive Diagnostics; Framingham, Mass.). After the enzymatic reaction was stopped by the addition of 2.5 N H₂SO₄, the optical density was read on an ELISA plate reader (Molecular Devices; Menlo Park, Calif.).

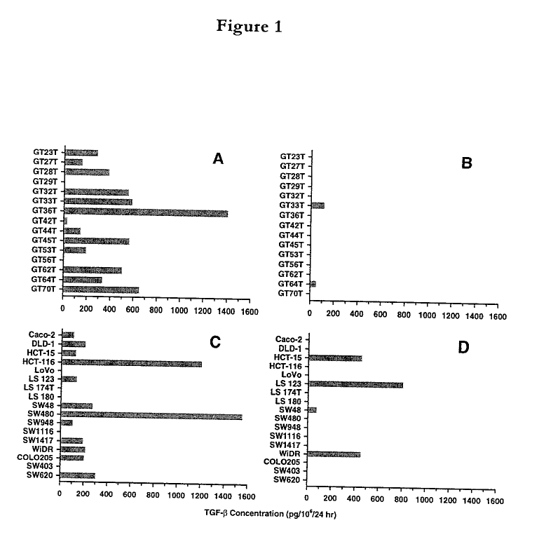

The results of TGF-β1 and TGF-β2 expression data are shown in FIG. 1. Thirteen of the 15 fresh colon carcinoma cultures (87%) secreted TGF-β1 compared to 12 of 16 (75%) established colon carcinoma cell lines. The range of TGF-β1 secretion was 0-1400 pg/10⁶ cells/24 hr for the fresh cultures and 0 to 1600 pg/10⁶ cells/24 hr for the established lines. Of the 15 cell lines established from colon carcinoma biopsies, 13 secreted TGF-β1 with a mean of 480 pg/10⁶ cells/24 hr and that ranged up to 1400 pg/10⁶ cells/24 hr. Fewer colon carcinoma cultures secreted TGF-β2 compared to TGF-β1. Two of the 9 (22%) fresh colon carcinoma cultures secreted TGF-β2 compared to 4 of 16 (25%) established colon carcinoma cell lines (FIG. 1). The cell lines SW620, COLO 205 and SW403 did not express detectable IL-10 or prostaglandins. Based on these criteria, COLO 205, SW620, and SW403 were identified as good candidates. As shown in Table 1, COLO 205 and SW620 secreted 200 and 300 pg TGF-β1/10⁶ cells/24 hr, while SW403 did not secrete measurable TGF-β1. None of the cells secreted measurable TGF-β2, IL-10, or prostaglandin-2.

TABLE 1
Secretion of Immunosuppressive Factors by Colon
Carcinoma Cell Lines

| | TGF-β1 | TGF-β2 | | |
|---|---|---|---|---|
| | (pg/10⁶ | (pg/10⁶ | | |
| Cells | cells/24 hr) | cells/24 hr) | IL-10 | PGE |
| COLO 205 | 200 | Neg | Neg | Neg |
| SW620 | 300 | Neg | Neg | Neg |
| SW403 | Neg | Neg | Neg | Neg |

The expression of the tumor antigens CEA, MUC-1, Ep-CAM, HER-2/neu, p53, and MAGE by fresh cultures and established colon carcinoma cell lines was evaluated. For analysis of tumor antigen expression, immunofluorescence flow cytometry was performed as previously described (Shawler et al., J. Clin. Lab. Anal., 1:184-190 (1987). To detect MHC Class I and Class II expression, direct immunofluorescence was performed using phycoerythrin-conjugated murine monoclonal anti-HLA-A,B,C and fluorescein-conjugated murine monoclonal anti-HLA-DR (Pharmingen; San Diego Calif.). For indirect immunofluorescence, the unconjugated murine monoclonal antibodies KS1/4 (anti-EpCAM; Lexigen Pharmaceuticals Corp.; Lexington, Mass.), anti-p53 (Clone DO-1; Calbiochem; San Diego, Calif.), anti-c-neu (Clone AB-5; Calbiochem) and anti-MUC1 (Clone HMPV; Pharmingen) were employed. Isotype-matched mouse mycloma proteins were used as negative controls and fluorescein-conjugated goat anti-mouse IgG antisera (Sigma Chemical Co.) was used as the secondary antibody. To detect CEA, indirection immunofluorescence was performed on ethanol-fixed cells using rabbit antisera to CEA (Dako A/S; Glostrup, Denmark) as the primary antibody and fluorescein-conjugated F(ab′)₂ swine anti-rabbit Ig (DAKO A/S) as the secondary antibody. Staining for KS1/4, MUC1, and CEA was performed on ethanol-fixed cells. Staining for HER2/new was performed on unfixed cells. All immunofluorescence samples were analyzed using a FACScaliber flow cytometer (Becton Dickinson).

The sequence of the expressed p53 message was performed by RT-PCR as previously described (Gjerset et al., Molecular Carcinogenesis, 14:275-285 (1995)). Briefly, total cellular RNA from approximately 5×10⁵ cells was reverse transcribed into cDNA in a 10 μl reaction. Flowing reverse transcription, the entire p53 coding sequence was PCR-amplified using appropriate 5′ and 3′ primers and 1 μl of the cDNA in 100 μl final reaction volume. After amplification, 1 μl of the product was amplified asymmetrically using a reverse to forward primer ration of 50:1, with primers chosen so as to amplify a 596 base internal fragment from codon 104 to 308. The asymmetric product was sequenced using a Sequenase kit (USB, Cleveland, Ohio) using forward primers from codon 104 and 210.

For PCR analysis of MAGE gene expression, total RNA was extracted from pellets containing 5×10⁶ cells using TRIZOL (Gibco BRL.; Gaithersburg, Md.). Flowing extraction, 2.5 μg RNA was treated with DNase and reverse transcribed into cDNA using oligo dT and the SuperScript Preamplification system containing SuperScript II reverse transcriptase (Gibco BRL) (Chomczynski and Sacchi, Anal. Biochem., 162:156-159 (1987)). After incubating at 95° C. for 10 minutes, the cDNA was subjected to 35 cycles of amplification (30 seconds at 94° C. dissociation, 30 seconds at 65° C. annealing, and 1 minute at 72° C. extension) (Mullis and Faloona, Methods Enzymol., 155:335-350 (1987)). The following PCR oligonucleotides were used to prime the MAGE PCR:

MAGE-1 forward 5′ CGG CCG AAG GAA CCT GAC CCA G 3′ (SEQ ID NO:1)

reverse 5′ CCG TTG GGT CAC TCC CAA GGT CG 3′ (SEQ ID NO:2)

MAGE-2 forward 5′ AAG TAG GAC CCG AGG CAC TG 3′ (SEQ ID NO:3)

reverse 5′ GTC TGG CGA AGA AGG AGA AG 3′ (SEQ ID NO:4)

MAGE-3 forward 5′ TGG AGG ACC AGA GGC CCC C 3′ (SEQ ID NO:5)

reverse 5′ CGT CCG GAG GAC TAT TAG CAG G 3′ (SEQ ID NO:6)

MAGE-4 forward 5′ GAG CAG ACA GGC CAA CCG 3′ (SEQ ID NO:7)

reverse 5′ CGG ACT GCG TCT CAG GAA 3′ (SEQ ID NO:8)

MAGE-6 forward 5′ TGG AGG ACC AGA GGC CCC C 3′ (SEQ ID NO:9)

reverse 5′ TGT CCG AAG GAC TAT TAG TAG GAC 3′ (SEQ ID NO:10)

MAGE-12 forward 5′ GGT GGA AGT GGT CCG CAT CG 3′ (SEQ ID NO:11)

reverse 5′ AAC GAT TTC TAG TCA CCT CCC G 3′ (SEQ ID NO:12)

The size of the PCR products were MAGE-1, 421, bp; MAGE-2, 316 bp; MAGE-3, 725 bp; MAGE-4, 446 bp; MAGE-6, 727 bp; and MAGE-12, 392 bp (De Plaen et al., Immunogenetics, 40:360-369 (1994)).

Cells were tested for expression of MHC Class I and Class II. All cells were positive for MHC Class I expression and negative for MHC Class II. However, Colo205 and SW403 could be induced to express Class II in the presence of interferon (IFN). Cytoplasmic expression of CEA was measured by immunofluorescence flow cytometry, and secreted CEA in cell culture supernatants was measured by ELISA. CEA was expressed by 67% (10 of 15) of fresh colon cancer cultures and by 4 of 5 established cell lines, including SW620, COLO 205, and SW403. Immunofluorescence flow cytometry was employed to evaluate MUC-1, Ep-CAM, and HER-2/neu expression.

Table 2 shows a comparison of the antigen expression profiles, as determined by flow cytometry. The vaccine cell lines IR804 (SW620/CD80), IR806 (Colo205/CD80), and SW403 all expressed EpCAM, HER2/neu, and CEA. The cell lines IR806 and IR804, but not SW403, expressed MUC1. These phenotypes are representative of antigen expression in tumor cell lines established from patient biopsies. Three normal fibroblast cell lines were included as negative controls for all the antigens. As with IR804 and IR806, the parental SW620 and Colo 205 cell lines expressed EpCAM, HER-2/neu and MUC-1.

TABLE 2
Expression of EpCAM, MUC1, HER2/neu, CEA and DNA
Aneuploidy by Colon Cancer Tumor Cell Lines.

| Cell Line | EpCAM | HER2/neu | MUC1 | CEA |
|---|---|---|---|---|
| Established | | | | |
| colon cancer lines: | | | | |
| IR304 | + | + | + | + |
| IR806 | + | + | + | + |
| SW403 | + | + | − | + |
| Tumor cell lines | | | | |
| established from | | | | |
| colon cancer | | | | |
| biopsies: | | | | |
| GT23T | + | + | + | − |
| GT36T | − | − | + | − |
| GT42T | + | + | + | + |
| GT45T | + | − | + | − |
| GT53T | − | + | + | − |
| GT101T | + | − | + | − |
| GT102T | + | + | | − |
| GT104T | + | + | + | − |
| GT107T | + | + | + | + |
| GT110T | + | − | + | − |
| GT111T | + | + | + | − |
| Fibroblast cell | | | | |
| lines established | | | | |
| from skin biopsies: | | | | |
| GT42F | − | − | − | − |
| GT53F | − | − | − | − |
| GT88F | − | − | − | − |

An anti-p53 monoclonal antibody was used to detect overexpression of p53 in 4 representative fresh colon carcinoma cultures. Nucleotide sequence analyses was then performed from amino acids 124 to 308 in 9 representative lines. Of the fresh colon carcinomas studied, 67% (6 of 9) demonstrated p53 abnormalities.

RT-PCR was used to determine the expression of the MAGE gene family members 1, 2, 3, 4, 6, and 12. The results of these assays are shown in Table 3. Each of these MAGE genes was expressed by at least one fresh colon cell culture. In the cell lines selected for the vaccine, IR806 and SW403 were PCR positive for MAGE-2, -3, -4, -6, and-12 expression but negative for MAGE-1. No amplification products were observed for any of the MAGE gene products in IR804. All MAGE antigens were expressed in at least one colon carcinoma cell line that had been established from patient biopsies, although MAGE-1 was expressed in only 2/12 lines tested. The most commonly expressed antigens were MAGE-4 (11 of 12), and MAGE-2 (9 of 12). MAGE-6 was expressed in 8 of 12 fresh colon cultures, and MAGE-6 and MAGE-12 were expressed in 7 of 12. Similar expression was seen in the 6 established colon lines tested. COLO 205 was the only colon cell line tested to not express MAGE mRNA.

TABLE 3
MAGE Expression in Colon Carcinoma Cell Lines

| Cells | MAGE-1 | MAGE-2 | MAGE-3 | MAGE-4 | MAGE-6 | MAGE-12 |
|---|---|---|---|---|---|---|
| Established colon | | | | | | |
| cancer lines: | | | | | | |
| IR804 | − | − | − | − | − | − |
| IR806 | − | + | + | ++ | + | ++ |
| SW403 | − | + | + | + | + | + |
| Tumor cell lines | | | | | | |
| established from | | | | | | |
| colon cancer | | | | | | |
| biopsies: | | | | | | |
| GT23T | + | − | ++ | + | − | + |
| GT42T | + | + | + | ++ | ++ | + |
| GT45T | − | ++ | ++ | ++ | +++ | +++ |
| GT50T | − | + | + | + | + | − |
| GT53T | − | − | − | + | − | − |
| GT54T | − | − | − | − | − | − |
| GT56T | − | + | − | + | − | − |
| GT62T | − | ++ | + | ++ | + | +++ |
| GTG4T | − | ++ | + | ++ | + | +++ |
| GT70T | − | ++ | + | ++ | + | +++ |
| GT71T | − | + | − | + | + | + |
| GT72T | − | + | − | + | + | − |

MAGE expression was also tested in the cell lines HCT-15, HCT-116 and SW480. HCT-15 was found to express MAGE-2 (+) and MAGE-4 (+); HCT-116 was found to express MAGE-2 (+), MAGE-3 (+++), MAGE-4 (+), and MAGE-6 (+); SW480 was found to express MAGE-2 (+), MAGE-3 (+), MAGE-4 (+), MAGE-6 (+), and MAGE-12 (+).

Based on the data presented in FIG. 1 and Tables 2 and 3, the cell lines SW620, COLO 205 and SW403 were chosen for further characterization and development as a potential whole cell vaccine for colon cancer. These cell lines are HLA-A2 positive, do not secrete high levels of immunosuppressive factors, and express a spectrum of putative tumor antigens representative of colon carcinomas. To further evaluate the expression of shared antigens by these cell lines, CTLs from a normal HLA-A2 positive donor were induced by stimulation with irradiated SW620 cells in a limiting dilution culture. A chromium release assay was used to test the resulting clones for cytotoxicity against the HLA-A2 positive cell lines SW620, the colon carcinoma cell line GT53T, the normal skin fibroblast cell line GT53F, which is isogenic to GT53T, and HT-29, an established HLA-A2 negative colon carcinoma cell line.

For generation of cytotoxic T-lymphocytes (CTL) and chromium release assay, CTLs were generated using a limiting dilution culture method. PBMC obtained from a normal HLA-A2 positive donor were incubated in 96-well flat-bottom plates (Costar Inc.) with 2×10⁴ irradiated (10,000 cGy) stimulator cells at effector:stimulator cell ratios of 5:1, 1.67:1, and 0.56:1. On day 4, human IL-2 and IL-4 (R&D Systems) were added to make final concentrations of 50 U/ml and 5 ng/ml, respectively. On days 7 and 14, the cells were restimulated with 2×10⁴ irradiated stimulator cells, 50 U/ml IL-2, and 5 ng/ml IL-4. On day 21, half of the cells were removed from each well, and the cells were tested for cytolytic activity by a standard chromium release assay using the stimulator cells as targets (Dillman et al., J. Immunol., 136:728-731 (1986)).

CTL clones were expanded by placing 1×10⁵ cells in a T25 flask (Costar, Inc.) with 2.5×10⁷ allogeneic PBMC irradiated at 3600 cGy, 5×10⁶ allogeneic EBV-transformed B cells irradiated at 10,000 cGy, 10 ng/ml anti-CD3 (Zymed; San Francisco, Calif.), 25 U/ml human IL-2, and 30 ml RPMI-1640 media (Mediatech, Inc.; Herndon, Va.) supplemented with 10% human serum (Gemini Bioproducts; Calabasas, Calif.). On days 5 and 8, 20 ml of media were removed and replaced with 20 ml fresh RPMI-1640 supplemented with 25 U/ml human IL-2 and 10% human serum. Cells were harvested for chromium release assay on days 12-14.

For the chromium release assay, 2×10⁶ target cells were labeled with 250 μCi Cr-51. After extensive washing, the target cells were placed in 96-well V-bottom plates (Costar Inc.) at a concentration of 1×10³ cells per well. One-third of the cells from each well were added to the target cells. The plates were centrifuged for 5 minutes at 100×g and were then incubated at 37° C. for 4 hours. Following the incubation, the plates were centrifuged at 500×g for 5 minutes and the Cr-51 radioactivity was measured in 100 μl aliquots of the supernatants. Background Cr-51 release was determined by incubating the target cells with 2.5 N H₂SO₄. The percent specific lysis was calculated using the formula (cpmₑₓₚ−cpm_{bkgd})/(cpmₘₐₓ−cpm_{bkgd})×100. Wells demonstrating >10% specific lysis were further expanded and were then tested for killing against a larger panel of target cells.

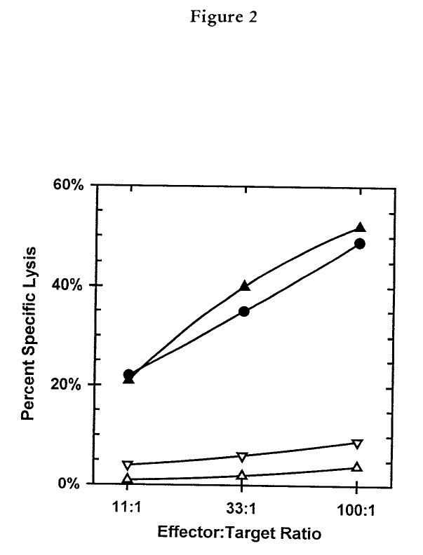

FIG. 2 shows HLA-A2-restricted cross-reactive cytoactivity induced by stimulation with SW620. The CTL lysed the HLA-a2 positive established colon carcinoma line SW620 on the HLA-A2 positive colon tumor GT53T. The CTL did not lyse the normal skin fibroblast line GT53F, which is isogenic to GT53T, or the HLA-A2 ne negative colon carcinoma line HT-29. Thus, the clone VP-5B8 demonstrated cytolytic activity against the two HLA-A2 positive colon lines, but not against the isogenic HLA-A2 positive fibroblast line or the HLA-A2 negative colon line.

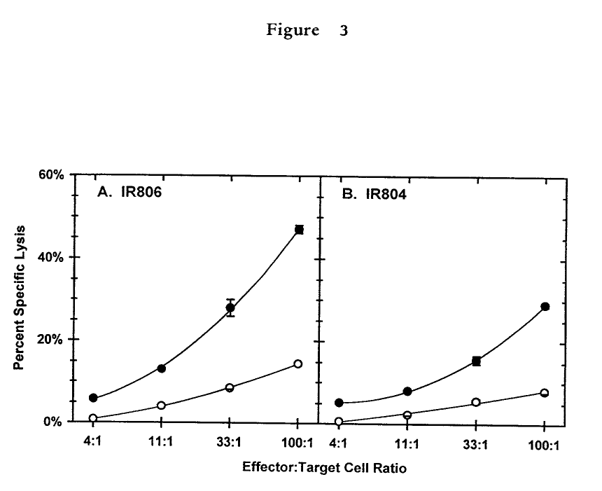

To test for stimulation of T Cells by CD80+tumor cells in vitro, the colon carcinoma cell lines COLO 205 and SW620 were genetically modified to constitutively express the costimulatory molecule CD80 (B7.1). Clones of these genetically modified cell lines were then tested for their ability to stimulate T cell responses from PBMC of normal, HLA-A2-positive individuals using a standard chromium release assay. IR804 (SW620/B7.1) and IR806 (COLO 205/B7.1) induced five to six fold greater tumor cytolytic activity compared to that obtained with parental SW620 and COLO 205 cells (FIG. 3). Thus, the CD80-expressing clones of SW620 and COLO 205 induced superior lytic activity compared to the parental lines.

To further evaluate the expression of shared antigens by these tumor cell lines, CTLs from a normal HLA-A2 positive donor were induced by stimulation with irradiated IR806 cells in a limiting dilution culture. A chromium release assay was used to test the resulting clones for cytotoxicity against IR806. Positive clones were retested for cytotoxicity against IR806 with and without inhibitory anti-Class I antibodies. Positive clones which were inhibited by the anti-Class I antibody were expanded for further analysis. Clones that were sufficiently expanded were tested for cytotoxicity against IR806, the HLA-A2 positive colon carcinoma cell line GT53T, a normal skin fibroblast cell line, GT53F, which is isogenic to GT53T, and HT-29, an HLA-A2 negative colon carcinoma cell line. FIG. 2 shows that a representative clone, VP-5B8, demonstrated cytolytic activity against the two HLA-A2 positive colon lines, but not against the isogenic HLA-A2 positive fibroblast line or the HLA-A2 negative colon line. These data indicate that VP-5B8 and similar clones recognize shared tumor-associated antigens in the context of HLA-A2 expression.

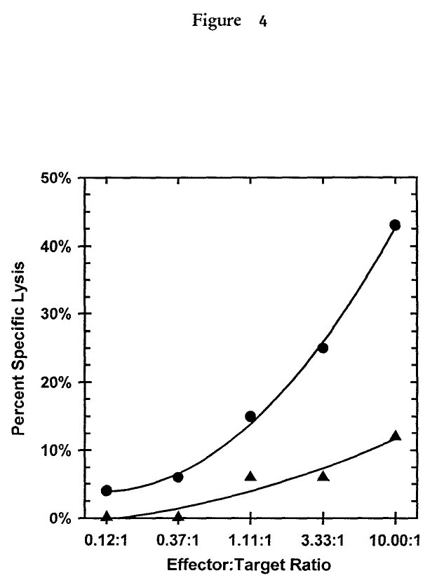

To test whether the cells comprising the tumor cell vaccine presented known TAAs in an HLA-A2 restricted fashion, we used p53 as the TAA and determined whether a p53-specific CTL clone could recognize p53 presented by SW620, one of the vaccine components that is known to overexpress p53 (Nigro et al., Nature 342:705 (1989); Rodrigues et al., Proc. Natl. Acad. Sci. USA 87:7555 (1990)). The wild type amino acid sequence 264-272 of p53 was previously identified as an immunodominant, HLA-A2.1 restricted peptide (Theobald et al., Proc. Natl. Acad. Sci. (USA), 11993-11997 (1995)). CTL directed against this peptide lyse human HLA-A2.1 tumor cell lines that overexpress p53 but do not lyse cells with normal p53 levels. An HLA-A2 restricted anto-p53 CTL clone was tested for cytolytic activity against SW 620 target cells using a standard chromium release assay. SW620, which overexpresses p53, was lysed by CTL A2 264, clone 15, which is an HLA-A2.1 CTL clone specific for p53 264-272 (FIG. 4). As a negative control, SW620 was not lysed by an HLA-A2.1 clone specific for the HIV pol 9K antigen. These data demonstrate that SW620 presents this immunodominant epitope of p53 in an HLA-A2 restricted manner.

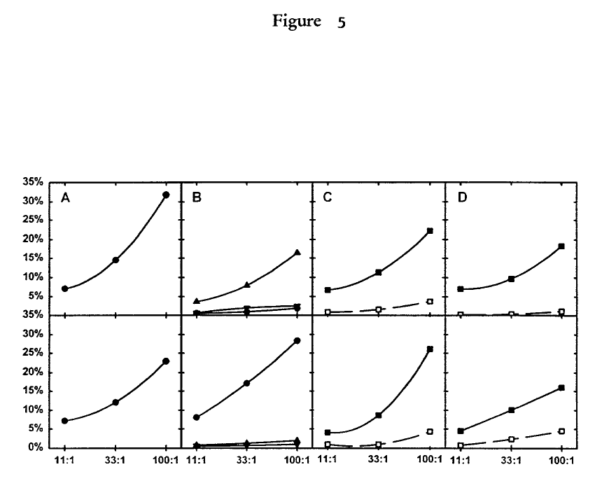

In order for an effective immune responses to ensue from vaccination with allogeneic cell lines, the CTLs generated via vaccination should recognize the patient's own tumor. A clinical trial has recently been completted in which HLA-A2 positive patients with advanced colon cancer were vaccinated with these cell lines and IL-2 secreting fibroblasts. Clones derived from the limiting dilution analysis of the immunized patient's CTL response were characterized. The data from two representative clones is shown in FIG. 5. Each clone was reactive to the respective autologous tumor cell line (Panel A). In addition, each clone showed a specificity for one the immunizing lines (Panel B), suggesting the presence of restricting colon specific antigens. Further evidence of the colon carcinoma specific nature of these clones was observed by their ability to lyse other A2.l colon cell lines but not their matched isogenic fibroblast lines (Panels C and D).

These results demonstrate that several cell lines have characteristics suitable for inclusion in an allogeneic colon cancer vaccine. The cell lines SW620, COLO 205, and SW403, are MHC HLA-A2 positive and collectively express the following shared TAAs: CEA, MUC-1, Ep-CAM, HER-2/neu, p53, and MAGE 2, 3, 4, 6, and 12. None of the selected cell lines secreted high levels of TGF-β1, and these cells did not express the immunosuppressive factors TGF-β2, IL-10 or prostaglandins. These results demonstrate that allogeneic tumor cells can stimulate MHC-restricted CTL that recognize shared TAAs and that genetic modification of the tumor cell lines to express CD80 increased their ability to stimulate CTL.

### EXAMPLE II

### Protocol for Phase I Clinical Trial of an Allogeneic Tumor Cell Vaccine

This example describes the protocol used to test the effect of allogeneic tumor cells genetically modified to express B7.1 (CD80) mixed with allogeneic fibroblasts genetically modified to secrete IL-2 in patients with colorectal carcinoma.

A Phase I clinical trial was completed in colon cancer patients using immunogene therapy comprising multiple administrations of autologous tumor cells and autologous fibroblasts genetically modified to express IL-2. In this dose escalation study, three patients were treated in cohorts where the dose of IL-2 was 100, 200 and 400 units of IL-2 secreted by the genetically modified fibroblasts. An additional patient was treated with an 800 unit dose of IL-2 secreting fibroblasts. All patients received the same level of tumor cells, held constant at 10⁷ cells. No treatment related toxicities of significance were observed. Delayed type hypersensitivity skin reactions (DTH) were observed in five of 10 patients and fatigue or flu like symptoms were experienced by 8 of 10 treated patients. It is important to note that DTH can be a positive sign of immune responses elicited. Clinically, stable disease of 12 weeks duration was observed in one patient. Progressive disease following vaccination was observed in the remaining patients.

Cytotoxic T cell precursor (pCTL) frequency analyses were performed to measure cell mediated immunity in a subset of these patients with sufficient cells for evaluation. The patients' autologous tumor cells (ATC) were utilized as stimulator cells, and pre and post treatment peripheral blood mononuclear cells were employed as effector cells in these assays. As a control to measure general immune competence, precursor frequencies were concurrently measured against allogeneic peripheral blood mononuclear cells (allo-PBMC). Low frequencies of tumor pCTL (range=1/190,000 to 1/1,320,000 PBMC) were detected prior to therapy in 4 of 6 patients. There was a 5 fold increase following treatment in the frequency of tumor pCTL in 2 of 3 evaluable patients with detectable pre-treatment pCTLs. The responding patients (#5 and #8) were treated with fibroblasts secreting 200 and 400 units of IL-2 per 24 hours, respectively. Cloned T cells derived from their corresponding precursor cultures were cytotoxic for tumor cells but not autologous fibroblasts. The allogeneic precursor frequency was unaffected by therapy in these patients. Patients #1 and #7 did not have detectable pre-treatment pCTL, and there was no increase in tumor pCTL frequency with treatment. Neither patient treated at the 100 unit dose of IL-2 (#1 and #3) showed increased pCTL. In patient #3, the precursor frequencies for autologous tumor and allogeneic pCTL decreased with time, implying worsening general immunosuppression with tumor progression.

For testing the allogeneic tumor cell vaccine, the study design was an open label, phase I, single center, multiple dose, dose escalating trial of allogeneic tumor cell lines genetically modified to express B7.1 mixed with allogeneic fibroblasts genetically modified to secrete IL-2 in patients with metastatic colon cancer. The allogeneic tumor cell dose was constant at 6×10⁷ irradiated tumor cells (2×10⁷ cell per each line). The fibroblasts genetically modified to express IL-2 were dose escalated to provide 0, 400 and 4000 BRMP units of IL-2 per 24 hrs. The patients received 3 intradermal immunizations at Weeks 0, 2 and 4. Twelve patients were enrolled, 4 in each dose group.

In the treatment of colon cancer patients with autologous tumor cells and IL-2 secreting autologous fibroblasts, delayed type hypersensitivity skin reactions were observed at injection sites without other significant side effects up to the maximum administered IL-2 dose of 800 units per 24 hrs. In an animal model, these doses of IL-2 were effective in generating systemic anti-tumor immunity. Therefore, therapy was initiated with transfected fibroblasts that secrete approximately this dose of IL-2. The transfected fibroblasts express approximately 3,000 units of IL-2 per 106 cells per 24 hours. Thus, approximately 5×10⁵ transfected fibroblasts were administered for the initial immunizations. The dose of IL-2 secreting cells was escalated as indicated in Table 4.

TABLE 4
Dose Escalation of IL-2

| | | Tumor | | Approximate # |
|---|---|---|---|---|
| | # of | Cell | Units of | of |
| Group | Patients | Dose¹ | IL-2² | Fibroblasts³ |
| 1 | 4 | 6 × 10⁷ | 0 | 0 |
| 2 | 4 | 6 × 10⁷ | 400 | 1.33 × 10⁵ |
| 3 | 4 | 6 × 10⁷ | 4000 | 1.33 × 10⁶ |

¹Patients received 6 × 10⁷ allogeneic modified tumor cells comprising a mixture of 2 × 10⁷ cells of each of 3 HLA-A2 positive colon carcinoma cell lines SW 620, Colo 205, and SW403. SW620 and Colo 205 were genetically modified to express B7.1.
²Biological Response Modifier Program (BRMP)
³Fibroblasts express approximately 3,000 BRMp units of IL-2 per 10⁶ cells per 24 hours.

The dose of transfected fibroblasts was escalated when 4 patients at each dosage level were treated without ≧grade 3 toxicity, for a total of 12 patients if no toxicity was observed. If 3 patients at the same dosage level developed unacceptable treatment related toxicity (≧grade 3 toxicity), the study was continued at the previous dose level until 3 additional patients were treated. The treatment of additional patients at the lower dosage level permitted further assessment of the effects of transduced cell injections at the lower dose. These parameters defined the Phase I study.

If significant anti-tumor effects were observed in a given patient, for example, partial response, further treatment was given if additional cells were available for immunization. These additional cycles of immunizations were administered at the discretion of the investigators, as warranted by the demonstration of anti-tumor immune response and improvement in the patient's clinic status.

A total of twelve patients meeting the inclusion and exclusion criteria described below were enrolled in the study. All patients were enrolled in the study through the Sidney Kimmel Cancer Center (SKCC) prior to starting therapy.

To be eligible for therapy, the patient fulfilled the following criteria: (1) Male or female, 18 years or older; (2) Histologically confirmed metastatic colorectal carcinoma with measurable disease. Imaging studies by enhanced Computerized Tomography (CT) or Magnetic Resonance Imaging (MRI) must be performed within two weeks of treatment initiation; (3) failed standard therapy with a 5-fluorouracil based regimen prior to initiation of treatment; (4) expected survival of at least three (3) months; (5) Karnofsky score of greater than or equal to 60%; (6) Baseline hematology and chemistry studies within two (2) weeks of treatment to meet the following values: (a) hemoglobin >9.0 gm/dl; (b) total granulocyte count >2000/mm³; (c) platelet count >100,000/m³; (d) BUN (blood urea nitrogen) <30 mg/dl; (e) creatinine <2 mg/dl; (f) alkaline phosphatase <4× upper limit of normal; (g) SGOT <4× upper limit of normal; (h) amylase <1× upper limit of normal; (i) lipase <lx upper limit of normal; (j) PT (prothrombin), PTT (partial thromboplastin time) <1.5× normal; (7) have a positive reaction to 2 of 7 antigens of the Merieux skin test panel within one month of treatment initiation; (8) HLA-A2 positive; (9) CT guided tumor biopsy or clinically indicated surgical tumor resection; (10) have ability to give informed consent.

Patients meeting the following exclusion criteria were excluded from the study; (1) Pregnant or lactating females; (2) the following medical conditions are exclusionary: known HIV, Hepatitis C or persistent Hepatitis B infection, autoimmune disorders (including ulcerative or granulomatosis colitis), hepatic cirrhosis, heart disease >New York Association III, or myocardial infarction within 6 months; (3) no tumor treatments four (4) weeks before initiation of treatment; (4) No treatment with immunosuppressive agents.

The Phase I clinical trial was directed to several objectives. One objective was to evaluate the safety of multiple intradermal immunizations of an allogeneic colon cancer vaccine containing three allogeneic tumor cell lines genetically modified to express B7.1 mixed with allogeneic fibroblasts genetically modified to secrete IL-2. Two of the three tumor cell lines were genetically modified to express B7.1. Another objective was to determine active biological and maximum tolerated dose of irradiated IL-2 transduced fibroblasts by examining increasing doses of the genetically modified fibroblasts. Still another objective was to evaluate the immunogenicity of this allogeneic cell line vaccine by measuring the level of cellular and humoral anti-tumor immune responses induced by the immunizations. Finally, an objective of the study was to examine the effects of the immunizations on tumor growth.

This open label, three arm trial involved the administration of irradiated tumor cells at a dose of 6×10⁷ cells (2×10⁷ cells/line) and fibroblasts expressing IL-2 at doses of 0, 400 and 4000 BRMPs (Table 4). The patients receiving the zero BRMP IL-2 dose did not receive any fibroblasts. The colon cancer vaccine was administered on Weeks 0, 2, and 4 (days 0, 14 and 28). The vaccine was administered intradermally as two 0.25 ml injections for a total volume of 0.5 ml.

A total of 12 patients were enrolled. The first group of 4 patients received 6×10⁷ irradiated tumor cells and no fibroblasts. After each injection, patients were examined and monitored for two hours and contacted daily for three days. At Weeks 0, 2 and 4, patients were examined and immunized. Patients were evaluated at study weeks 6 and 8 (2 and 4 weeks after the third immunization) and were seen monthly thereafter for 3 months (Weeks 12, 16 and 20). Long term safety was evaluated by asking the patient to return to the clinic every 12 weeks (approximately every 3 months) during the first year (Weeks 32, 44 and 56) and then yearly thereafter.

The dose of transfected fibroblasts was escalated when 4 patients at each dosage level were treated without ≧grade 3 toxicity. If no toxicity was observed, 12 patients were be treated. If 3 patients at the same dosage level developed unacceptable treatment related toxicity (≧grade 3 toxicity), the study was continued at the previous dose level until 3 additional patients were treated. The treatment of additional patients at the lower dosage level permitted further assessment of the effects of transduced cell injections at the lower dose.

The study procedures are outlined in Table 5.

TABLE 5
Study Outline of Phase I Colon Cancer Study of
Allogeneic Colon Cancer Vaccine

| | | Study Weeks | | | | | | | |
|---|---|---|---|---|---|---|---|---|---|
| | Screen | 0 | 2 | 4 | 6 | 8 | 12 | 16 | 20 |
| Medical History | X | | | | | | | | |
| HLA Typing | X | | | | | | | | |
| Merieux Skin Test | X | | | | | | | | |
| Physical Exam | X | X | X | X | X | X | X | X | X |
| Vital Signs | X | X | X | X | X | X | X | X | X |
| Karnofsky | X | | | | | X | | | |
| Medications | X | X | X | X | X | X | X | X | X |
| Adverse Events | | X | X | X | X | X | X | X | X |
| Tumor Biopsy | X | | | | | | | | |
| Hematology | X | X | X | X | X | X | X | X | X |
| Chemistry | X | X | X | X | X | X | X | X | X |
| Urinalysis | X | X | X | X | X | X | X | X | X |
| Pregnancy Test | X | X | X | X | X | X | X | X | X |
| PT, PTT | X | X | X | X | X | X | X | X | X |
| CEA | X | X | X | X | X | X | X | X | X |
| MRI/Cat Scan | X | | | | | X | | X⁴ | |
| Humoral Assays³ | | X | X | X | X | X | X⁴ | X⁴ | X⁴ |
| Cellular Assays³ | | X | X | X | X | X | X⁴ | X⁴ | X⁴ |
| Biopsy of | | | | | | X² | | | |
| Immunization Site | | | | | | | | | |
| Immunizations¹ | | X | X | X | | | | | |

¹Patients were monitored for 2 hours after each immunization and contacted daily for 3 days.
²Biopsy of site was taken 48 hours after the third immunization.
³Humoral assays: Collected 1 red top tube (10 mL). Cellular assays: Collected 1 red top tube and 4 green top tubes (50 mL).
⁴Optional dependent on patient's medical progress.

For screening, the following steps were performed: (a) obtained informed consent; (b) obtained medical history; (c) performed physical examination, including vital signs; (d) assessed Karnofsky status; (e) obtained blood samples within 2 weeks of treatment initiation for (i) hematology, chemistry, PT, PTT, CEA and urinalysis profiles and (ii) samples for humoral and cellular assays; (f) obtained MRI/CT scan demonstrates measurable disease; (g) administered Merieux skin test (skin test to be evaluated and read after 48 hours); (h) confirmed biopsy sample obtained.

At week 0 (Day 0), pre-immunization period, the following steps were performed: (a) performed physical examination and vital signs; (b) obtained blood samples for (i) hematology, chemistry, PT, PTT, CEA and urinalysis profiles (Appendix III) and (ii) blood samples for humoral and cellular assays; (c) informed patient to return to hospital and contact investigator if they developed inflammation at the injection site or experienced fever, rash, arthralgia, edema or dyspnea.

At Week 0 (Day 0), post-immunization period, the following steps were performed: (a) recorded vital signs every 30 minutes for 2 hours; (b) discharged patient to home if medically warranted.

At Days 1, 2 and 3 (Post-Immunization), the patient was contacted daily to evaluate the injection site and elicit adverse effects.

At Weeks 2 and 4 (Second and Third Immunization), the following steps were performed: (a) performed physical examination and vital signs; (b) obtained blood samples for hematology, chemistry, PT, PTT, CEA and urinalysis profiles; (c) informed patient to return to hospital and contact investigator if they developed inflammation at the injection site or experienced fever, rash, arthralgia, edema or dyspnea; (d) recorded vital signs every 30 minutes for 2 hours; (e) discharged patient to home if medically warranted.

At Days 1, 2 and 3 (Post Week 2 and 4 Immunization), the patient was contacted daily to evaluate the injection site and elicit adverse effects.

At Weeks 6, 8, 12, 16 and 20, the following steps were performed: (a) performed physical examination and vital signs; (b) obtained blood samples for (i) hematology, chemistry, PT, PTT, CEA and urinalysis profiles and (ii) blood samples for humoral and cellular assays.

For long term follow-up every 3 months x 1 year, then yearly thereafter: (a) performed physical examination and vital signs; (b) obtained blood samples for (i) hematology, chemistry, PT, PTT, CEA and urinalysis profiles and (ii) blood samples for humoral and cellular assays if medically warranted.

For the Phase I clinical trial, three allogeneic colon tumor cell lines, SW620, Colo 205, SW403, were selected for inclusion in the vaccine preparation. The use of three lines rather than one tumor cell line was to increase the total number of different tumor antigens presented. Each of these cell lines expressed the MHC haplotype HLA-A2. To further increase their potential immunogenicity, two of the three tumor cell lines, SW620 and Colo 205, were genetically modified to express the co-stimulatory molecule CD80 (B7.1) by transfection with a CD80 vector.

The other component of the vaccine preparation was the immortalized embryonic (allogeneic) fibroblast cell line KMST-6, which was genetically modified to secrete IL-2 by transfection with an IL-2 vector. The use of an allogeneic fibroblast cell line had practical advantages obviating the need to generate a customized autologous fibroblast cell line for each patient. The plasmid vector, pKIM-kan utilized in this study and described in more detail below was similar to the vectors employed by a number of investigators for in vivo studies including recently approved investigations with human subjects.

Standard lipofection and electroporation techniques were utilized to transfect the tumor and fibroblast cell lines with the B7.1 (CD80) and IL-2 vectors, respectively. The IL-2 vector transfected KMST-6 fibroblasts and the B7.1 (CD80) vector transfected colon tumor cell lines were washed and then grown in culture media containing G418 (a neomycin analogue) to select for transfected cells expressing the neo^{R} gene and desired transgenes. The transfected KMST-6 fibroblasts were tested for expression of the IL-2 gene by measurements of the concentration of IL-2 in the culture supenatant by an enzyme-linked immuno-absorbent assay (ELISA). The transfected tumor cells were tested for CD80 expression by immunofluorescence flow cytometry to confirm satisfactory genetic modification by the CD80 vector. The transfected cells were expanded in culture media containing G418/hygromycin and stored in treatment sized aliquots at −70° C. until required.

For preparation of irradiated cells, the cells were centrifuged and washed in DMEM media and then cryopreserved in a solution containing 10% dimethyl sulphoxide and 27% fetal calf serum in DMEM media. The cells were stored in liquid nitrogen until the time of administration. The transfected tumor cells utilized for immunizations were treated with 10,000 rads as described for the adjuvant active immunotherapy trial of colon cancer with autologous tumor (Hoover et al., J. Clin Oncol., 11:390-399 (1993)), and resuspended in a normal saline solution prior to injection. Transfected fibroblasts were irradiated with 4,000 rads to minimize the risk of chronic local inflammatory reactions at the site of immunization due to continued secretion of IL-2. This dose of radiation was shown to render fibroblasts incapable of proliferation while having no significant effect on the short term level of IL-2 secretion.

The vaccine was administered intradermally as two 0.25 mL inoculations for a total of 0.5 mL. The vaccine was administered in the deltoid region of the arm. The opposite arm (deltoid region) was used for each subsequent immunization. Patients received these immunizations on Weeks 0, 2 and 4.

Local toxicity at the sites of cell administration was treated with either topical steroids and/or surgical excision of the injection site as deemed appropriate. Patients were monitored for hypersensitivity reactions, mild chills, fever and/or rash and were treated symptomatically with antipyretics and antihistamines, if appropriate. Patients were not treated prophylactically.

If arthralgias, lymphadenopathy or renal dysfunction occurred, the investigators were notified and treatment with corticosteroids and/or antihistamines were instituted, if appropriate. Although anaphylactoid type hypersensitivity reactions were not anticipated, anaphylaxis was treated by administration of epinephrine, fluids, steroids and cardiopulmonary support, if appropriate. Standard guidelines were followed for any anaphalaxis treatment, including administration of epinephrine, maintaining airway passage, administration of oxygen, administration of aminophylline or P-agonist to treat bronchospasm, volume expansion by administration of saline or Ringer's solution, administration of a vasopressor to manage hypertension, administration of corticosteroids for serious or prolonged reactions, or delaying absorption of antigen by applying a tourniquet.

The maximum tolerated dose (MTD) was defined as that dose of IL-2 secreting cells which results in >grade 3 toxicity or which causes a delay in immunization in ≧3 patients in any treatment cohort. Immunization treatments were delayed if any ≧grade 2 toxicity was not reversed to ≦grade 1. If 3 patients at the same dosage level developed unacceptable treatment related toxicity (≦grade 3 toxicity), the study continued at the previous dose level until 3 additional patients were treated. The treatment of additional patients at the lower dosage level permitted further assessment of the effects of transduced cell injections at the lower dose. A dose limiting toxicity (DLT) was defined as any ≧grade 3 toxicity according to the NCI toxicity scale (Table 6). Dose limiting toxicities with the tumor cell vaccine was not anticipated based on previous findings.

TABLE 6
NCI Common Toxicities
GRADE¹

| TOXICITY | 0 | 1 | 2 | 3 | 4 |
|---|---|---|---|---|---|
| White Blood | 4.0 | 3.0-3.9 | 2.0-2.9 | 1.0-1.9 | <1.0 |
| Count | | | | | |
| Platelets | WNL² | 75.0 - WNL | 50.0-74.9 | 25.0-49.9 | <25.0 |
| Hemoglobin | WNL | 10.0 - WNL | 8.0-10.0 | 6.5-7.9 | <6.5 |
| Granulocytes/ | 2.0 | 1.5-1.9 | 1.0-1.4 | 0.5-0.9 | <0.5 |
| Bands | | | | | |
| Lymphocytes | 2.0 | 1.5-1.9 | 1.0-1.4 | 0.5-0.9 | <0.5 |
| Hemorrhage | None | Mild, no | Gross, 1-2 units | Gross, 3-4 units | Massive, >4 |
| (Clinical) | | transfusion; | transfusion per | transfusion per | units per |
| | | petechiae | episode | episode | episode |
| Infection | None | Mild | Moderate | Severe | Life threatening |
| Nausea | None | Able to eat; | Intake | No significant | |
| | | reduced but | significantly | intake | |
| | | reasonable | decreased but | | |
| | | intake | still can eat | | |
| Vomiting | None | 1 episode in 24 | 2-5 episodes in | 6-10 episodes in | >10 episodes in |
| | | hours | 24 hours | 24 hours | 24 hours, or |
| | | | | | requiring |
| | | | | | parenteral |
| | | | | | support |
| Diarrhea | None | Increase of 2-3 | Increase of 4-6 | Increase of 7-9 | Increase of ≧10 |
| | | stools/day over | stools/day, or | stools/day or | stools/day or |
| | | pre-Rx | nocturnal | incontinence, or | grossly bloody |
| | | | stools, or | severe cramping | diarrhea, or |
| | | | moderate | | need for |
| | | | cramping | | parenteral |
| | | | | | support |
| Skin | None or | Scattered | Scattered | Generalized | Exfoliative |
| | no | macular or | macular or | symptomatic | dermatitis or |
| | change | papular eruption | papular eruption | macular, | ulcerating |
| | | or erythema that | or erythema with | papular, or | dermatitis |
| | | is asymptomatic | pruritus or the | vesicular | |
| | | | associated | eruption | |
| | | | symptoms | | |
| Local | None | Pain | Pain and | Ulceration | Plastic surgery |
| | | | swelling with | | indicated |
| | | | inflammation or | | |
| | | | phlebitis | | |
| Hand-Foot | No | Mild | Moderate | Painful swelling | Not applicable |
| Syndrome | symptoms | paresthesias | paresthesias | of distal | |
| | | +/or numbness of | +/or numbness | phalanges with | |
| | | fingers +/or | with or without | or without local | |
| | | toes | local dermatitis | dermatitis | |
| Stomatitis | None | Painless ulcers, | Painful | Painful | Requires |
| | | erythema, or | erythema, edema, | erythema, edema, | perenteral or |
| | | mild soreness | or ulcers, but | or ulcers and | enteral support |
| | | | can eat | cannot eat | |
| Bilirubin | WNL | — | <1.5 × N | 1.5-3.0 × N | >3.0 × N |
| Transaminase | WNL | ≦2.5 × N | 2.6-5.0 × N | 5.1-20.0 × N | >20.0 × N |
| (SGOT, SGPT) | | | | | |
| Alk Phos or | WNL | 2.5 × N | 2.6-5.0 ×N | 5.1-20.0 × N | >20.0 × N |
| 5′ Nucleo- | | | | | |
| tidase | | | | | |
| Liver- | No | — | — | Precoma | Hepatic coma |
| Clinical | change | | | | |
| | from | | | | |
| | baseline | | | | |
| Creatinine | WNL | <1.5 × N | 1.5-3.0 N | 3.1-6.0 × N | >6.0 × N |
| Proteinuria | No | 1+ or <0.3 g % or | 2-3+ or 0.3- | 4+ or | Nephrotic |
| | change | <3 g/l | 1.0 g % or 3-10 g/l | >1.0 g % or 10 g/l | syndrome |
| Hematuria | Neg | Micro only | Gross, no clots | Gross | Requires |
| | | | | clots | transfusion |
| Alopecia | No loss | Mild hair loss | Pronounced or | — | — |
| | | | total hair loss | | |
| Pulmonary | None or | Asymptomatic, | Dyspnea | Dyspnea at | Dyspnea at rest |
| | no | with abnormality | on significant | normal level of | |
| | change | in PFT's | exertion | activity | |
| Cardiac: | None | Asymptomatic, | Recurrent or | Requires | Requires |
| dysrhythmias | | transient | persistent, no | treatment | monitoring; or |
| | | requiring no | therapy required | | ventricular |
| | | therapy | | | tachycardia or |
| | | | | | fibrilla-tion |
| Cardiac: | None | Asypmtomatic | Asymptomatic | Mild CHF | Severe or |
| function | | decline of | decline of | responsive to | refractory CHF |
| | | resting ejection | resting ejection | therapy | |
| | | fraction by less | fraction by more | | |
| | | than 20% of | than 20% of | | |
| | | baseline value | baseline value | | |
| Cardiac: | None | Non-specific T- | Asymptomatic ST | Angina without | Acute infarction |
| ischemia | | wave flattening | and | evidence for | |
| | | | T-wave changes | infarction | |
| | | | suggesting | | |
| | | | isohemia | | |
| Cardiac: | None | Asymptomatic | Pericarditis | Symptomatic | Tamponade; |
| pericardial | | effusion, no | (rub, chest | effusion; | drainage |
| | | intervention | pain, ECO | drainage | urgently |
| | | required | changes) | required | required |
| Hypertension | None or | Asymptomatic | Recurrent or | Requires therapy | Hypertensive |
| | no | transient | persistent | | crisis |
| | change | | increase >20 mm | increase >20 mm | |
| | | Hg (Dia) or to | Hg (Dia) or to | | |
| | | >150/100 if BP | >150/100 if BP | | |
| | | previously nl. | previously nl. | | |
| | | No treatment | No treatment | | |
| | | required. | required. | | |
| Hypotension | Noneor | Changes | Requires fluid | Requires therapy | Requires therapy |
| | no | requiring no | replacement or and | and | |
| | change | therapy | other therapy | hospitalization | hospitalization |
| | | (including | but not | resolves within | for >48 hours |
| | | transient | hospitalization. | 48 hours of | after stopping |
| | | orthostatic | | stopping the | the agent |
| | | hypotension) | | agent | |
| Neuro: | None or | Mild | Mild or moderate | Severe objective | — |
| sensory | no | parethesias, | objective | sensory loss or | |
| | change | loss of deep | sensory loss; | pares-thesias | |
| | | tendon reflexes | moderate pares- | that interfere | |
| | | | thesias | with function | |
| Neuro: motor | None or | Subjective | Mild objective | Objective | Paralysis |
| | no | weakness; no | weakness without | weakness with | |
| | change | objective | significant | impairment of | |
| | | findings | impairment of | function | |
| | | | function | | |
| Neuro: | None | — | Simple partial | Complex partial | Seizures of any |
| seizures | | | seizures in | seizures with | type which are |
| | | | which conscious- | altered | prolonged, |
| | | | ness is | conscious-ness | repetitive or |
| | | | preserved; self- | or generalized | difficult to |
| | | | limited and/or | seizures with | control (status |
| | | | controlled | loss of | epilepticus) |
| | | | | consciousness; | |
| | | | | self-limited | |
| | | | | and/or | |
| | | | | controlled | |
| Neuro: | None | Mild somnolence | Moderate | Severe | Coma, seizures, |
| cortical | | or agitation | somnolence or | somnolence, | toxic psychosis |
| | | | agitation | agitation, | |
| | | | | confusion, | |
| | | | | disorientation | |
| | | | | or | |
| | | | | hallucinations | |
| Neuro: | None | Slight | Intention | Locomotor ataxia | Cerebellar |
| cerebellar | | incoordina-tion, | tremor, | | necrosis |
| | | dysdiado-kinesis | dysmetria, | | |
| | | | slurred speech, | | |
| | | | nystagmus | | |
| Neuro: mood | No | Mild anxiety or | Moderate anxiety | Severe anxiety | Suicidal |
| | change | depression | or depression | or depression | ideation |
| Neuro: | None | Mild | Moderate or | Unrelenting and | — |
| headache | | | severe but | severe | |
| | | | controllable | | |
| Neuro: | None or | Mild | Moderate | Severe | Ileus >96 hrs. |
| constipation | no | | | | |
| | change | | | | |
| Neuro: | None or | Asypmtomatic | Tinnitus | Hearing loss | Deafness not |
| hearing | no | hearing loss on | | interfering with | correctable |
| | change | audiometry | | function but | |
| | | | | correct-able | |
| | | | | with hearing aid | |
| Neuro: vision | None or | — | Blurred vision | Symptomatic | Blindness |
| | no | | or diplopia | subtotal loss of | |
| | change | | | vision | |
| Allergy | None | Transient rash | Urticaria, drug | Serum sickness | Anaphylaxis |
| | | or drug fever | fever 38° C. | or broncho- | |
| | | 38° C. (100.4° F.) | (100.4° F.), mild | spasm; requires | |
| | | | broncho-spasm | parenteral | |
| | | | | medication | |
| Fever in | None | 37.1-38° C. (98.7- | 38.1-40.0° C. | >40.0° C. | >40.0° C. |
| absence of | | 100.4° F.) | (100.5-104.0° F.) | (>104.0° F.) for | (>104.0° F.) for |
| infection | | | | less than 24 | 24 hours, or |
| | | | | hours | fever with |
| | | | | | hypotension |
| Fatigue | No | Performance | Performance | Performance | Performance |
| | change | score (ECOC) | score (ECOG) | score (ECOG) | score (ECOC) |
| | in | decrease in 1 | decrease in 2 | decrease in 3 | decrease to PS 4 |
| | perform- | level, but not | levels, but not | levels, but not | |
| | ance | to PS 4 | to PS 4 | to PS 4 | |
| | score | | | | |
| | (ECOG) | | | | |
| | and not | | | | |
| | PS 4 | | | | |
| Chills | None | Chilly | Mild rigors, no | Severe rigors, | Not applicable |
| | | sensation, no | medication | requires | |
| | | rigors | required | medication | |
| Myalgias | None | Mild muscular | Moderate aching | Severe muscular | Not applicable |
| | | aching; no | requiring | aching requiring | |
| | | medication | medication; | no medication; | |
| | | required | associated | associated | |
| | | | enzyme | enzyme (CPK) | |
| | | | (CPK) elevation | elevation | |
| Weight | <5.0% | 5.0-9.9% | 10.0-19.9% | >20.0% | |
| gain/loss | | | | | |
| Hyper- | <116 | 116-160 | 161-250 | 251-500 | >500 or |
| glycemia | | | | | ketoacidosis |
| Amylase | WNL | <1.5 × N | 1.5-2.0 × N | >2.1-5.0 × N | >5.1 × N |
| Hyper- | <10.6 | 10.6-11.5 | 11.6-12.5 | 12.6-13.5 | >13.5 |
| calcemia | | | | | |
| Hypocalcemia | >8.4 | 8.4-7.8 | 7.7-7.0 | 6.9-6.1 | ≦6.0 |
| Hypo- | >1.4 | 1.4-1.2 | 1.1-0.9 | 0.8-0.6 | ≦0.5 |
| magnesemia | | | | | |
| Fibrinogen | WNL | 0.99-0.75 × N | 0.74-0.50 × N | 0.49-0.25 × N | ≦0.24 × N |
| Prothrombin | WNL | 1.01-1.25 × N | 1.26 × 1.50 × N | 1.51-2.00 × N | >2.00 × N |
| time | | | | | |
| Partial | WNL | 1.01-1.66 × N | 1.67-2.33 × N | 2.34-3.00 × N | >3.00 × N |
| thrombo- | | | | | |
| plastin time | | | | | |

For management of toxicities, immunization treatments were delayed if any ≧grade 2 toxicity was not reversed to ≧grade 1. In cases where toxicities of grade 2 or 3 were felt by the investigator to be not related or remotely related to therapy, treatment continued with appropriate monitoring according to standard medical practice.

Adverse events were monitored and reported if appropriate. Subjects were instructed by the investigator to report the occurrence of any adverse clinical event. An adverse event was any undesirable event associated with the use of a drug, whether or not considered drug related, and included any side effect, injury, toxicity, or sensitivity reaction. It also included any undesirable clinical or laboratory change which did not commonly occur in the subject.

A complete listing of blood tests used are described in Table 5. All laboratory reports were reviewed by the investigator or his/her designate. The investigator commented on all laboratory values outside the specified normal range. All laboratory values verified as abnormal and clinically significant were documented as adverse laboratory values on the appropriate page of the case report form (CRF). If repeat testing confirmed a value within acceptable ranges, the value was documented as normal.

A serious adverse event was one that was fatal or life-threatening, required inpatient hospitalization, prolonged hospitalization, or was disabling (or was permanently or severely disabling. Death, congenital anomaly, cancer or overdose was always considered a serious event. Progression of a subject's underlying condition leading to one of the above was reported as a serious (but expected) adverse event.

“Life threatening” indicated that the subject was at immediate risk of death from the event as it occurred. It did not include an event that, had it occurred in a more serious form, might have caused death. “Requires inpatient hospitalization” was defined as hospital admission required for treatment of the adverse event. Hospital admission for scheduled elective surgery was not considered a serious adverse event. If the adverse event was serious, it was reported within 24 hours by telephone to Dr. Robert E. Sobol at the Sidney Kimmel Cancer Center. The serious adverse event also was reported in writing within two days to Dr. Robert E. Sobol, The Sidney Kimmel Cancer Center, 10835 Altman Row, San Diego, Calif. 92121. Withdrawal from the study and therapeutic measures were determined per the protocol. A full explanation of discontinuation from the study was made on the appropriate case report form. All adverse events, regardless of severity, were followed up by the investigator until satisfactory resolution.

Adverse clinical or laboratory events were graded according to the NCI Common Toxicity Criteria described in Table 6. If the adverse event was not found in the NCI Common Toxicity table, the following ratings were utilized (Table 7):

TABLE 7
Adverse Event Rating - Non-NCI Common Toxicity

| | |
|---|---|
| (1) Mild | Generally non-progressive; transient or |
| | mild discomfort; no limit&ion in activity, |
| | no medical intervention/therapy required. |
| (2) Moderate | Mild to moderate limitation in daily |
| | activity; some assistance needed; no or |
| | minimal medical intervention/therapy |
| | required. |
| (3) Severe | Marked limitation in daily activity; some |
| | assistance usually required; medical |
| | intervention/therapy required. |
| (4) Very Severe | Extreme limitation in daily activity; |
| | significant assistance required; |
| | significant medical intervention/therapy |
| | required. |

For discontinuation criteria, subjects could withdraw from this study or treatment of their own volition or, if in the judgment of the treating physician, withdrawal from treatment was medically or psychologically indicated. Specific reasons for treatment discontinuation included: (a) development of grade ≧3 toxicity; (b) patient's tumor failed to respond to treatment (progressive disease); (c) patient had completed therapy; (d) patient refused further therapy (e) patient developed other medical problems unrelated to the study that precluded further therapy. Specific reasons for study discontinuation were: (a) disease progression; (b) death; (c) initiation of non-protocol treatment.

Patients who were off treatment but not off study continued to be followed for disease progression/response, late reports of toxicity and survival. Once a patient was off study because of disease progression or initiation of non-protocol treatment, the patient was followed for survival purposes.

Immunological and clinical response criteria were monitored. Humoral anti-tumor and anti-vaccine immune responses were evaluated by flow cytometry. Briefly, autologous tumor, KMST-IL-2 fibroblasts, and the three colon carcinoma cell lines used for vaccination were incubated with 3 fold serial dilutions of serum from treated subjects to determine if antibodies to the tumor or the components of the vaccine were generated. Binding of specific antibodies to the cell surface was assessed using a fluorscein conjugated anti-human IgG reagent. The intensity of staining and titer of the sera pre- and post-vaccination were compared.

For Cytotoxic T Lymphocyte (CTL) precursor frequency analysis, precursor frequency analyses of cytotoxic effector cells were performed using a previously described limiting dilution method (Coulie et al., International J. Cancer 50:289-297 (1992)). Briefly, 10⁴ irradiated target cells were mixed with various numbers of effector cells in 96 well V-bottom plates and cultured in media supplemented with IL-4 (5 units/ml). IL-2 (30 units/ml) was added to the cultures on day 3. The cultures were re-fed on day 7 with fresh media containing IL-4 (5 units/ml) and IL-2 (30 units/ml) and 104 irradiated target cells. On day 14 the cells were harvested and employed in a standard chromium release assay. Precursor frequencies were estimated by Poisson distribution analysis and the X² minimization analyses described above (Coulie et al., supra 1992).

For skin biopsy of immunization site, standard hematoxylin and eosin staining and immunohistochemical methods employing monoclonal antibodies to hematopoetic cell subsets were employed to characterize the immune cell infiltrates observed in skin biopsies at immunization sites. Monoclonal antibodies to T-cells (CD2, CD3, CD4, CD8), natural killer cells (CD16, CD57, CD58) and B-cells (CD19, CD20) were employed for these studies. Briefly, for the immunohistochemical studies, cryostat sections were fixed in cold acetone and then incubated with the primary antibody 1 hour at room temperature. The sections were washed and then incubated with horseradish peroxidase conjugated secondary antibody followed by staining with an appropriate chromagen substrate and examined by light microscopy. Incubation of sections with isotype-matched control antibody instead of the primary antibody was utilized as a negative control.

An evaluation of the tumor response was performed one month after the third immunization. Tumor size was calculated by measuring the tumor's largest cross sectional area as measured by the greatest cross sectional diameter times the greatest diameter perpendicular to it on enhanced CT or MRI scans. Technical parameters for the evaluation scan were identical to those used for the baseline scan.

Response was defined as summarized below: Complete Response (CR): Disappearance of all measurable or evaluable disease, signs, symptoms and biochemical changes related to the tumor, for a minimum of 4 weeks, during which time no new lesions appeared. At the time of suspected CR, the patient was reevaluated completely, short of invasive procedures. Equivocal Complete Response (Equiv. CR): This rating was assigned where there was a complete response, but examination remained abnormal in such a way as to preclude an unequivocal statement that the tumor had completely disappeared. Partial Response (PR): This rating was assigned where there was a reduction by at least 50% of the product of the sum of the two longest perpendicular diameters of the lesion(s) without the appearance of new lesions lasting a minimum of 4 weeks. A bone response consisting of recalcification of lytic bone metastasis was included as partial response providing there was no disease progression elsewhere. Minor Response (MR): This rating was assigned where there was a reduction of less than 50% of the product of the two longest perpendicular diameters of the lesion(s) without the appearance of new lesions for a minimum of 4 weeks, and either no change in or partial recalcification of all osteolytic lesions. Objectively Stable (S): This rating was assigned where there was less than 25% increase in the product of the two longest perpendicular diameters of the lesion(s) without the appearance of new lesions. Progressive Disease (P): This rating was assigned where there was an increase of greater than or equal to 25% in the product of the two longest perpendicular diameters of the lesion(s) or the occurrence of a new lesion(s).

The protocol used was a Phase I study to assess the safety of active tumor immunotherapy with irradiated HLA-A2 matched allogeneic tumor cells genetically modified to express the co-stimulatory molecule B7.1 (CD80) mixed with irradiated allogeneic fibroblasts genetically modified to secrete IL-2. The goal was to obtain information concerning a maximum tolerated dose and an active biological dose of IL-2 gene therapy with genetically modified allogeneic fibroblasts. Outcomes were categorical (such as proportions developing toxicity or obtaining complete or partial remissions), or measurements of durations (such as time to relapse or total survival). A maximum tolerated dose was determined by assessments of toxicity utilizing the toxicity grading criteria of the National Cancer Institute (Table 6).

Preliminary data on the optimum biological dose was obtained by measurements of anti-tumor immune responses, complete and partial remission rates, time to progression and survival. Previous studies in animal models suggested that the generation of anti-tumor immunity was induced with lower levels of cytokine gene expression. Hence, the maximum tolerated dose and the optimum biological dose were not similar.

One goal of these studies was to identify the maximum tolerated dose (MTD) in this Phase I study and to obtain initial data on the optimum biological dose and relative response rates in patients with colorectal cancer. If the proposed dose escalation levels does not result in significant toxicity and if a higher biological dose is thought to be useful, permission is sought from regulatory agencies to further escalate the dose of IL-2 secreting fibroblasts to define the MTD and to further characterize effective biological doses which induce cellular and/or humoral anti-tumor immune responses. Information obtained from the Phase I trial will assist in the design of formal Phase II protocols where optimum biological doses and efficacy profiles will be established.

Prior to any procedures being performed at the screening evaluation, the subject was informed of the nature of the product being studied and was given pertinent information as to the intended purpose, possible benefits, and possible adverse events of the study. The procedures and possible hazards to which the subject was exposed were explained. An approved informed consent statement then was read and signed by the subject, a witness, and a member of the study team. The subject was provided with a copy of the informed consent statement. Verification of a signed informed consent statement was noted on the subject's case report form and medical source document. If the subject was legally incompetent, the written consent of a parent, legal guardian or legal representative was obtained.

The final approved protocol and the informed consent statement administered were reviewed by a properly constituted Ethics Committee or Institutional Review Board (IRB). Institutional Review Boards adhered to the Food and Drug Administration Regulations (21 CFR, Part 56). The investigator was responsible for obtaining initial and continuing review (at intervals no less than once a year) of the study by an IRB. The investigator agreed to make required progress reports to the Ethics Committee/IRB, as well as report any serious adverse events, life-threatening problems or death. The investigator also informed the Ethics Committee/IRB of reports of serious adverse events.

For preparation of vaccine components, the cell lines used in the allogeneic cell vaccine are described below and summarized in Table 8.

TABLE 8
Summary of Cell Lines

| Cell Lines | Description |
|---|---|
| KMST-6/IL-2 | KMST-6 is a human fibroblast cell line |
| | genetically modified to express IL-2 |
| SWG20-B7.1 | Colon cancer cell line genetically |
| | modified to express B7.1 (CD80) |
| Colo 205/B7.1 | Colon cancer cell line genetically |
| | modified to express B7.1 (CD80) |
| SW403 | Colon cancer cell line |

The KMST-6/IL-2 cell line was derived from the KMST-6 cell line, which is a human fibroblast line that has undergone immortilization by repeated exposure to ⁶⁰Co radiation. Its parental cell line, KMST-6, was derived from the eight week old embryo of a healthy 28 year old Japanese woman. The karyotype of these cells was normal with no structural abnormalities. Once established, the cells were exposed to ⁶⁰Co gamma rays thirteen times over 197 days for a total of 2800 rads. The resulting transformed cell line was designated KMST-6. Characteristics of this cell line Namba et al., include a B-type isozyme pattern of glucose-6-phosphate dehydrogenase (G6PD), a lactate-dehydrogenase isozyme pattern of human origin, and no evidence of infection by HSV-1, HSV-2, SV-40, or EBV, using immunofluorescent antibody techniques. In addition, no virus particles were observed by electron microscopy. The cell line also failed to form tumors upon transplantation into nude mice.

The rationale for selecting KMST-6 as a component for this vaccine instead of other embryonic fibroblast lines like MRC-5 or -9, or WI-38 was based on its immortalized growth characteristics. It was found that, with the other fibroblast lines, there were too few population doublings left in the life of the cell to establish a stable transfectant. Having a finite lifespan, these cells would senesce before a clone could be established, expanded, and frozen as a master cell bank. However because KMST-6 is able to undergo many more, if not unlimited population doublings, a stable transfectant could be master cell banked, ensuring consistency of manufacture.

In addition, KMST-6 has been used in another clinical trial. A clinical trial conducted in Germany in which 15 cancer patients received at least three injections of 2×10⁶ interleukin-2 (IL-2) gene modified KMST-6 cells formulated with 2×10⁶ autologous tumor cells. The injections were administered subcutaneously and intradermally at four week intervals. It was concluded the vaccine was safe and well tolerated (Veelken et al., Int. J. Cancer, 70:267-277 (1997)).

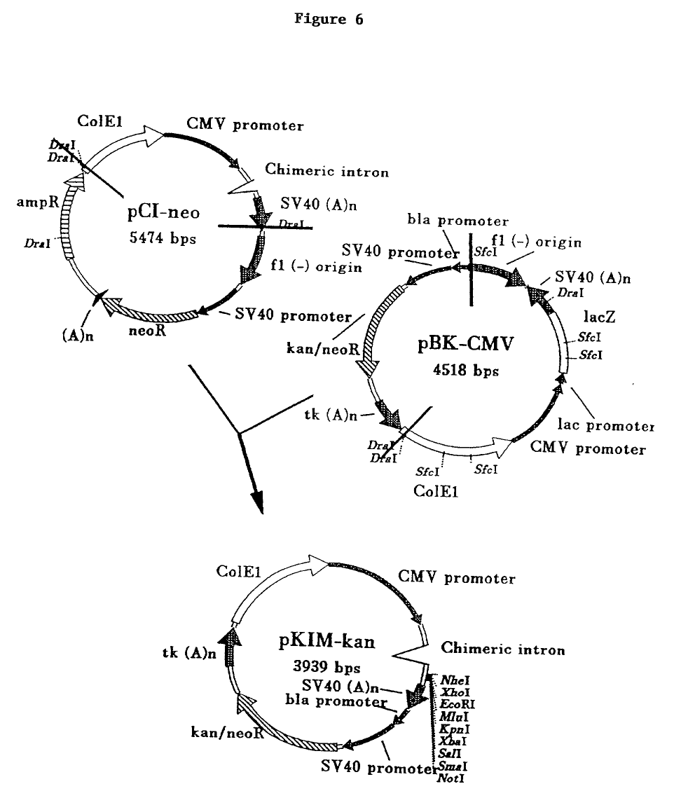

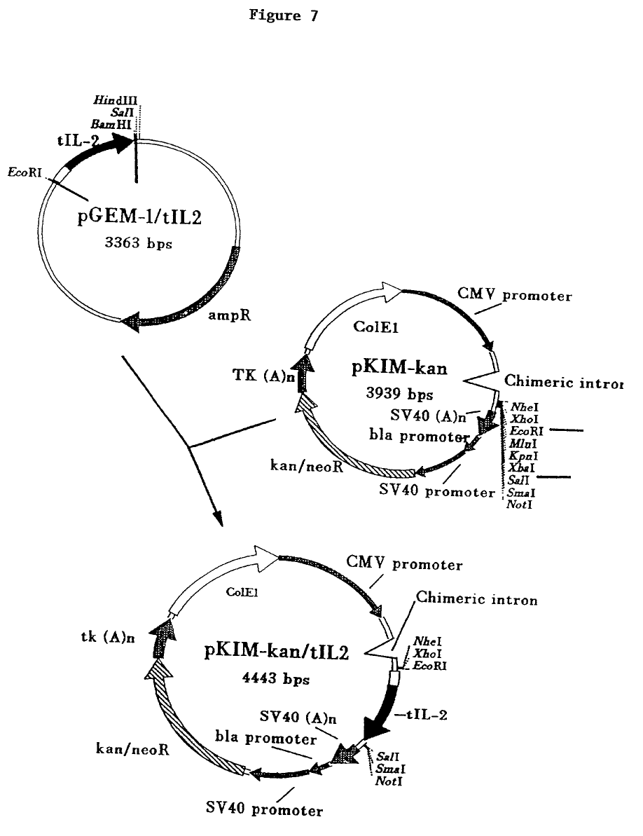

The pKIM-kan construct was made as shown in FIG. 6. For KMST-6 transfection with pKIM/kanl IL-2 vector, the parental KMST-6 cell line (provided by Dr. Masayoshi Namba) was transfected by electroporation with pKIM-kan/tIL-2, an expression plasmid containing the gene for human IL-2 (see FIG. 7). The surviving cells were placed under selection and cloned by the limiting dilution technique. Clones were screened by ELISA for their abiity to secrete IL-2 and a high producer KMST-6/IL-2 clone was chosen for use in this clinical trial.

For irradiation of KMST-6/IL-2 cells, the effects of different doses of radiation on cellular proliferation and IL-2 secretion were examined. In order to identify the amount of radiation required to inhibit proliferation of cell growth, 5×10^{KMST-}6 cells were irradiated with 0, 1000, 2000, and 4000 cGy, plated in T75 flasks, and evaluated for colony formation 14 days later. The results, shown in Table 9, demonstrate that 2000 cGy was the minimum radiation dose necessary to completely inhibit proliferation. Therefore, the dose of 4000 cGy was chosen for radiation of the KMST-6/IL-2 to be utilized in the vaccine preparation.

TABLE 9
Effect of Irradiation on Cell Proliferation

| | Radiation Dose | Number of |
|---|---|---|
| Cell Line | (cGy) | Colonies |
| KMST-6 | 0 | 120,000 |
| | 1000 | 11,000 |
| | 2000 | 0 |
| | 4000 | 0 |

Based on these data, IL-2 secretion by KMST-6/IL-2 fibroblasts that were irradiated at 4000 cGy, which is twice the dose required to inhibit cell proliferation, were tested. The results of IL-2 secretion measured by ELISA are presented in Table 10. Under these conditions, irradiated KMST-6/IL-2 secreted 2933±10⁶ U IL-2/10⁶ cells/24 hours after 24 hours and 2646+35 U IL-2/10⁶ cells/24 hours seven days after radiation. Non-irradiated KMST-6/IL-2 secreted 3748±608 U IL-2/10⁶ cells/24 hours. Thus, irradiation at 4000 cGy had only modest effects on IL-2 secretion by KMST-6/IL-2 cells.

TABLE 10
IL-2 Secretion by KMST-6/IL-2 Following
Irradiation with 4000 cGy

| | Units per 10⁶ | Percent of |
|---|---|---|
| Radiation Dose | cells per 24 hrs | Control |
| Control (non-irradiated) KMST- | 3748 ± 608 | |
| 6/IL-2 | | |
| KMST-6/IL-2; 1 day post | 2933 ± 106 | 78% |
| irradiation (4000 cCy) | | |
| KMST-6/IL-2; 7 days post | 2646 ± 35 | 71% |
| irradiation (4000 cGy) | | |

To generate the SW620/B7.1 cell line, the SW620 cell line (ATCC CCL 227), which was isolated from a lymph node metastasis from a 51 year old Caucasian male with Duke's stage C colorectal carcinoma, was used. The cells are epithelial in morphology, and are positive for keratin expression by immunoperoxidase staining. The cells grow attached in a monolayer. They are tumorigenic in nude mice, and are negative for reverse transcriptase. SW620 cells are aneuploid and express Class I, HLA-A2, ICAM, CEA and the c-myc, K-ras, H-ras, N-ras, myb, sis, and fos oncogenes (Leibovitz et al., Cancer Res., 36:4562-4569 (1976); Fogh et al., J. Natl. Cancer Inst., 58:209-214 (1977); Fogh et al., J. Natl. Cancer Inst., 59:221-226 (1977); Leibovitz et al., J. Natl. Cancer Inst., 63:635-645 (1979); Trainer et al., Int. J. Cancer, 41:287-296 (1988); Rodrigues et al., Proc. Natl. Acad. Sci. USA, 87:7555-7559 (1990)). The cells are negative for CSAp and colon antigen 3. The cells have a G to A mutation in codon 273 of the p53 gene resulting in an Arg to His substitution. The cells secrete 300 pg TGF-β1 per 10⁶ cells per 24 hrs but do not secrete the immunosuppressive factors TGF-β2 or IL-10.

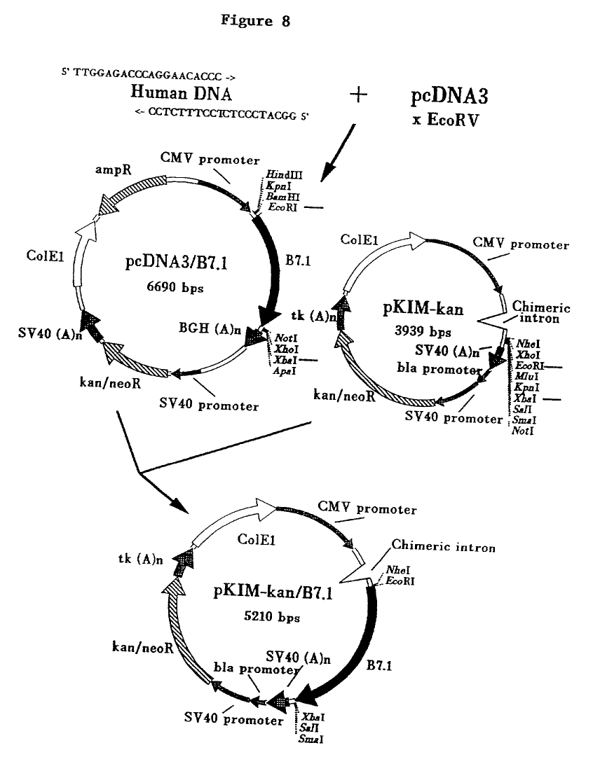

For SW620 transfection with KIM/kanB7.1 vector (FIG. 8), SW620 cells were obtained from the ATCC. The cells were transfected to express the human B7.1 cell surface protein (CD80) by electroporation with pKIMkan/B7.1. The surviving cells were placed under selection and cloned by the limiting dilution technique. Clones were screened by cytofluorometric analysis using a rhodamine-conjugated anti-human B7.1 murine antibody to detect surface expression of the B7.1 protein, and a single clone was selected for generating the Master Cell Bank for use in the clinical trial.

The COLO 205 cell line (ATCC CCL-222) was isolated from ascites fluid collected from a 70 year old Caucasian male with Duke's stage D colorectal carcinoma. The cells are epithelial in morphology, and are positive for keratin expression by immunoperoxidase staining. They grow loosely attached or in suspension. They are tumorogenic in nude mice, and are negative for reverse transcriptase. COLO 205 cells are diploid and express Class I, HLA-A2, ICAM, carcinoembryonic antigen (CEA), and a 36,000 Dalton cell surface glycoprotein related to the GA733-2 tumor associated antigen (Trainer et al., Int. J. Cancer, 41:287-296 (1988); Semple et al., Cancer Res., 38:1345-1355 (1978); Gastl et al., Int. J. Cancer, 55:96-101 (1993); Bjork et al., J. Biol. Chem., 268:24232-24241 (1993)). The cells are negative for CSAp and MAGE 1, 2, 3, 4, 6, 12. The cells are Class II negative, but become Class II positive following incubation with IFN-y. The cells secrete 200 pg TGF-β1per 10⁶ cells per 24 hrs but do not secrete the immunosuppressive factors TGF-β2 or IL-10,

For COLO 205 transfection with KIM-kan B7.1 vector, COLO 205 cells were transfected to express the human B7.1 cell surface protein (CD80) by lipofection with pKIM-kan/B7.1. COLO 205 cells (106) were transfected with 3 μg of pKIM-kan/B7.1 DNA using 10 μL of LipofectAMINE reagent. Ali the cells were distributed to 48 wells (two 24-weU plates) for selection with G418. The surviving cells were placed under selection and cloned by the limit dilution technique. Clones were screened by cytofluorometric analysis using a rhodamine-conjugated anti-human B7.1 murine antibody to detect surface expression of the B7.1 protein, and a single clone was selected for use in the clinical trial.

The SW403 cell line (ATCC CCL 230) was isolated from a primary tumor of a 51 year old Caucasian female with Duke's stage C colorectal carcinoma. The cells are epithelial in morphology and are positive for keratin M expression by immunoperoxidase staining. The cells grow attached in clumps. They are tumorigenic in nude mice, and are negative for reverse transcriptase. SW403 cells are diploid and express Class I, HLA-A2, ICAM, CEA and colon antigen 3 (Leibovitz et al., Cancer Res., 36:4562-4569 (1976); Fogh et al., J. Natl. Cancer Inst., 59:221-226 (1977)). The cells express MAGE 2, 3, 4, 6, and 12. The cells are negative for CSAp. The cells do not secrete the immunosuppressive factors TGF-β1, TGF-β2, or IL-10. SW403 cells were obtained from the ATCC for generating the Master Cell Bank to be utilized in the clinical trial. Despite numerous attempts by both electroporation and lipofection, the SW403 cell line was not transfectable with the pKIM-kan/B7.1 vector. Nevertheless, the cell line was included in the vaccine preparation to provide an additional source of colon tumor antigens.

In order to identify the amount of radiation required to inhibit proliferation of tumor cell growth, 5×10⁶, cells each of SW620/B7.1, COLO 205/B7.1, and SW403 were irradiated with 0, 1000, 2000, and 4000 cGy, plated in T75 flasks, and evaluated for colony formation 14 days later. The results, shown in Table 11 demonstrate that 2000 cGy was the minimum radiation dose necessary to inhibit proliferation of these cell lines.

TABLE 11
Effect of Irradiation on Tumor Cell
Proliferation

| | Radiation Dose | Number of |
|---|---|---|
| Cell Line | (cGy) | Colonies |
| SW620/B7.1 | 0 | 150,000 |
| | 1000 | 6110 |
| | 2000 | 0 |
| | 4000 | 0 |
| Colo205/B7.1 | 0 | 88,000 |
| | 1000 | 400 |
| | 2000 | 0 |
| | 4000 | 0 |

Based on these data, it was determined that 10,000 cGy was a sufficient radiation dose to inhibit proliferation of the colon cancer cell lines used in this vaccine. Similar studies were performed with the SW403 cell line to confirm the adequacy of the proposed 10,000 cGy dose. The effect of 10,000 cGy irradiation on the expression of CD80 by the two genetically modified cell lines was determined. SW620/B7.1 and COLO 205/B7.1 cells were irradiated with a total of 10,000 cGy per cell line. Following 24 hours of culture, the cells were assayed for B7.1 expression by cytofluorometric analysis using a fluorescein-conjugated anti-CD80 monoclonal antibody. The results demonstrated that 10,000 cGy had little effect on CD80 expression in either cell line. A fluorescein-conjugated mouse IgG2A myeloma protein was used as a negative control.

A master cell bank (MCB) was established for each cell line: KMST-6/lL-2, SW620/B7.1, COLO 205/B7.1 and SW403. Briefly, cells were expanded into multiple T225 flasks until a total cell population of approximately 2.5×10⁹ cells was achieved. The cells were then detached from the flasks with trypsin, washed with medium, and enumerated. The cells were pelleted by centrifugation and resuspended in freeze medium at a density of approximately 5×10⁶ cells per mL. The freeze medium consisted of Dulbecco's Modified Eagle's Medium (DMEM) with the addition of 27% fetal bovine serum (FBS) and 10% dimethyl sulfoxide (DMSO). The cell suspension was distributed in one mL aliquots to approximately 500 cryovials, each labeled with the MCB lot number. Each MCB vial was clearly labeled with a minimum of the following information: Lot number, clone identification and description. The cryovials were then transferred to Styrofoam boxes and placed at −70° C. for a minimum of 24 hours before being placed into liquid nitrogen storage tanks. Each MCB was stored in segregated areas of two liquid nitrogen tanks.

Table 12 summarizes the certification testing of the master cell bank and production lot employed for this study.

TABLE 12
Characterization of Master Cell Banks

| TEST | SPECIFICATION |
|---|---|
| Viability & Concentration | >75% of cells viable 1.0 × |
| | 10⁶ |
| IL-2 Levels (KMST-6/IL-2 | >1000 units of IL-2 |
| only) | |
| Sterility | Sterile |
| Endotoxin | Negative |
| Mycoplasma | Negative |
| In Vitro Viral Assay | Negative |
| (Adventitious Virus) | |
| In Vivo for Adventitious | Negative |
| Virus | |
| Tumorigenicity, In Vivo, | Negative |
| Soft Agarose | |
| Isoenzyme & Cytogenic | Complies with Published |
| Analysis | report (Human) |
| Transmission Electron | No viral particles detected |
| Microscopy of Cultured | |
| Cells | |
| Bovine Viruses | Negative |
| HTLV-I & II (human T-cell | Negative |
| leukiemia virus) | |
| Hepatitis B | Negative |
| EBV (Epstein-Barr Virus) | Negative |
| Cytomegalovirus | Negative |
| HHV-6 (human herpesvirus-6) | Negative |
| Reverse Transcriptase | Negative |
| HIV Assay (human | Negative |
| immunodeficiency virus) | |

The production of each lot of colon cancer vaccine began with the thawing of one vial from the MCB of each of the three colon tumor cell lines. The cells were propagated in DMEM supplemented with 10% FBS, and the cultures are kept segregated at all times until combined later in the process. The cells were then detached from the flasks with trypsin, washed with medium, and enumerated. The number of cells required from each of the colon cell lines was calculated by multiplying the number of doses by 2×10⁷ cells. This number was then divided by the cell count of the cell suspension to determine the volume of suspension required. Once this was performed for each line, the cells were combined and mixed in a sterile, pyrogen free container. They were then pelleted by centrifugation and resuspended in medium at a concentration of 3×10⁷ cells per mL. The cells were then irradiated with 10,000 cGy from a ⁶⁰Co source. The cells were again pelleted by centrifugation, washed three times, and enumerated. Finally, the cells were frozen in DMEM freeze medium, supplemented with 27% FBS and 10% DMSO, in aliquots of 1.8 mL, containing 6×10⁷ colon tumor cells. The cryovials were transferred to Styrofoam boxes and placed at −70° C. for a minimum of 24 hours before being placed into liquid nitrogen storage tanks.

The KMST-6/IL-2 cells were prepared in much the same way as the colon tumor cell lines. First, each lot was initiated with one vial of cells from the MCB. The cells were cultured in flasks located in a segregated area of the incubator. The cells were prepared for irradiation as described above, then exposed to 4,000 cGy of ⁶⁰Co radiation. Once irradiated, the cells were washed and frozen in DMEM freeze media in aliquots of 1.0 mL containing the number of cells necessary to deliver a specific dose of IL-2 (ie. 400 or 4000 units). The cryovials were transferred to Styrofoam boxes and placed at −70° C. for a minimum of 24 hours before being placed into liquid nitrogen storage tanks.

A single dose of the vaccine was prepared for administration by thawing and combining the contents of one vial of colon tumor cells and one vial of IL-2 secreting fibroblasts. One dose of the colon tumor vaccine consists of a mixture of 2×10⁷ cells from each of the colon tumor cell lines: SW620/B7.1, COLO 205/B7.1, SW403, and the appropriate number of IL-2 secreting fibroblasts for the 400 U or the 4000 U dose of IL-2. The cells were washed three times with saline to remove the cryopreservative medium (DMEM +27% FBS and 10% DMSO) in which they were stored. The washed cells were resuspended to a final volume of 0.5 mL in injectable grade saline and 0.25 mL was drawn into two separate syringes for intradermal injection. A negative gram stain of the final wash supernatant was required before injection. A sample of the final wash supernatant was cryopreserved for archival purposes and sent for sterility testing (aerobic, anaerobic, and fungal cultures).

### EXAMPLE III

### Stimulation of an Immune Response to Tumor Antigens in Patients Having Colorectal Carcinoma and Treated With an Allogeneic Tumor Cell Vaccine

This example describes the results of a Phase I clinical trial of patients having colorectal carcinoma and treated with genetically engineered allogeneic tumor cell line vaccines.

Prior to these studies, it was unknown whether allogeneic vaccines expressed shared tumor antigens capable of inducing immune responses against patients' tumors. A Phase I clinical study in HLA-A2 colon cancer patients with a semi-allogeneic HLA-A2 tumor cell lines vaccine genetically modified to express the molecule CD80 was performed (see Examples I and II). The vaccine included a combination of the three cell lines IR804(SW620/CD80), IR806(COLO205/CD80) and SW403.

In 4 of 5 patients tested initially, the results indicated that immunizations with the genetically engineered vaccine increased the frequency of tumor cytotoxic T cell precursors (pCTL) approximately 5 to 10 fold against HLA-A2 autologous and allogeneic tumors. There was no change in pCTL frequency against allogeneic peripheral blood mononuclear cells run as a control. These findings were equivalent to the results obtained following vaccination with genetically engineered autologous vaccine preparations. Cloned CTL from the vaccinated patients demonstrated lysis of multiple HLA-A2+ tumor cells but not isogenic normal fibroblasts, indicating specificity for shared HLA-A2 restricted tumor associated antigen epitopes. These results indicate that colon carcinomas express shared tumor antigens and that immunization with genetically modified semi-allogeneic tumor cells can enhance anti-tumor immune responses to autologous tumors.

In further analysis of this Phase I clinical trial, there was a significant increase in the frequency of CTLs against COLO 205 in 5 of 6 patients. There was also a concomitant increase in CTL against the autologous tumor in 2 of 3 evaluable patients, that is, in those from whom fresh tumor cell cultures were obtained. Significantly, the frequency of CTLs against allogeneic peripheral blood mononuclear cells (PBMCs) did not increase, and even decreased in 2 of 6 patients. Two CTL clones derived from post-vaccination PBMCs were characterized for reactivity against various target cells. Both clones killed autologous tumor and the vaccinating lines. The CTL clones also killed colon carcinoma cells derived from other patients, but not their isogenic fibroblasts.

The effect of administering the allogeneic tumor cell vaccine on precursor CTL frequencies is shown in Table 13. Precursor CTL frequencies were measured for autologous tumor cells (ATC), COLO 205, SW620, SW403 and allogeneic PMBC (Table 13). The patients in Group 1 were given no IL-2 and the patients in Group 2 were adminstered allogeneic fibroblasts expressing 400 units of IL-2 per 24 hours. Treatment was safely tolerated. A positive immune response was demonstrated in 7 of 8 patients. Of the four patients from whom autologous tumor cells (ATC) were obtained, three had an increase in anti-ATC precursor pCTL following treatment. The results are summarized in Table 14.

TABLE 13
Effect of Allogeneic Tumor Cell Vaccine on Precursor CTL Frequencies.

| | | | | | | |
|---|---|---|---|---|---|---|
| | Time | anti- | anti- | anti- | anti- | anti- |
| Patient | Point | ATC | COLO 205 | SW620 | SW403 | alloPBMC |

Group 1: no IL-2

| | | | | | |
|---|---|---|---|---|---|
| 1 | Pre | 1/442,000 | 1/1,319,000 | | 1/79,000 |
| | Wk 8 | 1/118,000 | 1/256,000 | | 1/97,000 |
| 2 | Pre | | 1/80,000 | | 1/129,000 |
| | Wk 8 | | 1/97,000 | | 1/156,000 |
| 3 | Pre | <1/1,319,000 | <1/1,319,000 | | 1/240,000 |
| | Wk 7 | <1/1,319,000 | <1/1,319,000 | | 1/440,000 |
| | Wk 19 | 1/740,000 | <1/1,319,000 | | <1/740,000 |
| 4 | Pre | | 1/300,000 | 1/102,000 | 1/120,000 |
| | Wk 8 | | 1/81,000 | 1/72,000 | 1/99,000 |

Group 2: 400 U/24 hr IL-2

| | | | | | | |
|---|---|---|---|---|---|---|
| 5 | Pre | 1/922,000 | 1/922,000 | | | 1/65,000 |
| | Wk 7 | 1/318,000 | 1/256,000 | | | 1/389,000 |
| 6 | Pre | | 1/1,085,000 | 1/700,000 | 1/203,000 | 1/50,000 |
| | Wk 6 | | 1/80,000 | 1/71,000 | 1/62,000 | 1/88,000 |
| 7 | Pre | <1/1,500,000 | <1/1,500,000 | | | 1/280,000 |
| | Wk 6 | <1/1,500,000 | 1/850,000 | | | 1/540,000 |
| 8 | Pre | | 1/1,370,000 | 1/848,000 | 1/299,000 | 1/110,000 |
| | Wk 6 | | 1/349,000 | 1/480,000 | 1/69,000 | 1/129,000 |

TABLE 14
Changes in Precursor Cytotoxic T Lymphocyte Frequencies

| | anti- | anti- | anti- | anti- | anti- |
|---|---|---|---|---|---|
| | ATC | COLO205 | SW620 | SW403 | alloPBMC |
| Group 1: no IL-2 | | | | | |
| Patient 1 | ↑ | ↑ | | | nc* |
| Patient 2 | | nc | | | nc |
| Patient 3 | ↑ | nc | | | ⇓ |
| Patient 4 | | ↑ | ↑ | nc | nc |
| Group 2: 400 U/24 hr IL-2 | | | | | |
| Patient 5 | ↑ | ↑ | ↑ | | ⇓ |
| Patient 6 | | ↑ | ↑ | ↑ | nc |
| Patient 7 | nc | ↑ | ↑ | ↑ | ⇓ |
| Patient 8 | | ↑ | ↑ | ↑ | nc |
| Group 3: 4000 U/24 hr IL-2 | ↑ | ↑ | ↑ | | nc |
| Patient 9 | | | | | |

* nc = no change

Further analysis of the Phase I clinical trial in colorectal cancer patients treated with irradiated HLA-A2.1 matched allogeneic tumor cells genetically modified to express the co-stimulatory molecule B7.1 (CD80) mixed with irradiated allogeneic fibroblasts genetically modified to secrete IL-2 was performed. Increases in cytotoxic T cell precursor frequencies (pCTL) to the immunizing lines were observed in the majority (7/9) of evaluable patients. Importantly, increases in pCTL frequencies to autologous tumor cells were induced in 4/5 evaluable patients. Several T cell clones expanded from the patients lysed autologous tumors and other HLA-A2⁺ colon tumor cells but not their isogenic fibroblasts. The vaccine was well tolerated in all patients without significant toxicity. These data demonstrate that allogeneic tumor cells can induce T cell immune responses to antigens shared by patients' tumors, indicating the usefulness of a genetically modified allogeneic tumor cell vaccine in colorectal cancer.

These results indicated that an allogeneic tumor cell vaccine comprising allogeneic tumor cell lines expressing tumor antigens increased the frequency of CTLs against the allogeneic cell lines and against autologous tumor cells of the patient. Furthermore, CTL clones also killed colon carcinoma cell derived from other patients. These data support continued clinical evaluation of the CD80 modified allogeneic colon carcinoma vaccine.

Throughout this application various publications have been referenced. The disclosures of these publications in their entireties are hereby incorporated by reference in this application in order to more fully describe the state of the art to which this invention pertains.

Although the invention has been described with reference to the examples provided above, it should be understood that various modifications can be made without departing from the spirit of the invention. Accordingly, the invention is limited only by the claims.

What is claimed is:

1. A composition for stimulating an immune response in a patient having an adenocarcinoma, comprising an allogeneic tumor cell selected from the group consisting of a SW620 cell, COLO 205 cell, and SW403 cell and a physiologically acceptable carrier.

2. The composition of claim 1, wherein said adenocarcinoma is selected from the group consisting of colon, breast, lung or prostate adenocarcinoma.

3. The composition of claim 1, further comprising an allogeneic cell genetically modified to express a cytokine.

4. The composition of claim 3, wherein said cytokine expressing allogeneic cell is a fibroblast.

5. The composition of claim 3, wherein said cytokine expressing allogeneic cell is a tumor cell.

6. The composition of claim 3, wherein said cytokine is interleukin-2 (IL-2) or granulocyte macrophage-colony stimulating factor (GM-CSF).

7. The composition of claim 6, wherein said cytokine expressing allogeneic tumor cell expresses membrane-bound GM-CSF.

8. The composition of claim 1, wherein said composition comprises a SW620 cell, COLO 205 cell, and SW403 cell.

9. The composition of claim 1, wherein at least one of said allogeneic tumor cells is genetically modified to express CD80 (B7.1).

10. The composition of claim 9, wherein said genetically modified cell is a SW620 cell or COLO 205 cell.

11. The composition of claim 10, wherein said composition comprises a SW620 cell and a COLO 205 cell genetically modified to express CD80 (B7.1).

12. The composition of claim 11, further comprising a SW403 cell.

13. A composition for stimulating an immune response in a patient having an adenocarcinoma, comprising a SW620 cell, a COLO 205 cell, and a SW403 cell, said SW620 and COLO 205 cells genetically modified to express CD80 (B7.1), and an allogeneic fibroblast cell genetically modified to express IL-2.

14. A composition for stimulating an immune response in a patient having colorectal cancer, comprising an allogeneic tumor cell selected from the group consisting of a SW620 cell, COLO 205 cell, and SW403 cell and a physiologically acceptable carrier.

15. The composition of claim 14, further comprising an allogeneic cell genetically modified to express a cytokine.

16. The composition of claim 15, wherein said cytokine expressing allogeneic cell is a fibroblast.

17. The composition of claim 15, wherein said cytokine expressing allogeneic cell is a tumor cell.

18. The composition of claim 17, wherein said cytokine is interleukin-2 (IL-2) or granulocyte macrophage-colony stimulating factor (GM-CSF).

19. The composition of claim 18, wherein said cytokine expressing allogeneic tumor cell expresses membrane-bound GM-CSF.

20. The composition of claim 14, wherein said composition comprises a SW620 cell, COLO 205 cell, and SW403 cell.

21. The composition of claim 14, wherein at least one of said allogeneic tumor cells is genetically modified to express CD80 (B7.1

22. The composition of claim 21, wherein said genetically modified cell is a SW620 cell or COLO 205 cell.

23. The composition of claim 22, wherein said composition comprises a SW620 cell and COLO 205 cell genetically modified to express CD80 (B7.1).

24. The composition of claim 23, further comprising a SW403 cell.

25. A composition for stimulating an immune response in a patient having colorectal cancer, comprising a SW620 cell, a COLO 205 cell, and a SW403 cell, said SW620 and COLO 205 cells genetically modified to express CD80 (B7.1), and an allogeneic fibroblast cell genetically modified to express IL-2.

26. A method of stimulating an immune response in a patient having an adenocarcinoma, comprising administering to said patient one or more allogeneic tumor cells, wherein at least one of said allogeneic tumor cells is selected from the group consisting of a SW620 cell, COLO 205 cell, and SW403 cell, whereby said allogeneic cell stimulates an immune response to an autologous tumor cell in said patient.

27. The method of claim 26, wherein said adenocarcinoma is selected from the group consisting of colon, breast, lung and prostate adenocarcinoma.

28. The method of claim 26, further comprising the step of administering an allogeneic cell genetically modified to express a cytokine.

29. The method of claim 28, wherein said cytokine expressing allogeneic cell is a fibroblast.

30. The method of claim 28, wherein said cytokine expressing allogeneic cell is a tumor cell.

31. The method of claim 28, wherein said cytokine is interleukin-2 (IL-2) or granulocyte macrophage-colony stimulating factor (GM-CSF).

32. The method of claim 31, wherein said allogeneic tumor cell is genetically modified to express membrane-bound GM-CSF.

33. The method of claim 26, wherein said immune response comprises a cytotoxic T lymphocyte (CTL) response.

34. The method of claim 26, whereby a CTL response to autologous non-tumor cells is minimized.

35. The method of claim 34, wherein said autologous non-tumor cells are peripheral blood mononuclear cells.

36. The method of claim 26, wherein at least one of said allogeneic tumor cells is genetically modified to express CD80 (B7.1).

37. The method of claim 36, wherein said genetically modified allogeneic tumor is SW620 or COLO 205.

38. The method of claim 37, wherein said one or more allogeneic tumor cells is a combination of SW620, COLO 205, and SW403.

39. A method of stimulating an immune response in a patient having an adenocarcinoma, comprising administering to said patient a composition comprising a SW620 cell, a COLO 205 cell, and a SW403 cell, said SW620 and COLO 205 cells genetically modified to express CD80 (B7.1), and an allogeneic fibroblast cell genetically modified to express IL-2, whereby said allogeneic cell stimulates an immune response to an autologous tumor cell in said patient.

40. A method of stimulating an immune response in a patient having colorectal cancer, comprising administering to said patient one or more allogeneic tumor cells, wherein at least one of said allogeneic tumor cells is selected from the group consisting of SW620, COLO 205, and SW403 and wherein said allogeneic cell stimulates an immune response to an autologous tumor cell in said patient.

41. The method of claim 40, further comprising the step of administering an allogeneic cell genetically modified to express a cytokine.

42. The method of claim 41, wherein said cytokine expressing allogeneic cell is a fibroblast.

43. The method of claim 41, wherein said cytokine expressing allogeneic cell is a tumor cell.

44. The method of claim 41, wherein said cytokine is interleukin-2 (IL-2) or granulocyte macrophage-colony stimulating factor (GM-CSF).

45. The method of claim 44, wherein said allogeneic tumor cell is genetically modified to express membrane-bound GM-CSF.

46. The method of claim 40, wherein said immune response comprises a cytotoxic T lymphocyte (CTL) response.

47. The method of claim 40, whereby a CTL response to autologous non-tumor cells is minimized.

48. The method of claim 47, wherein said autologous non-tumor cells are peripheral blood mononuclear cells.

49. The method of claim 40, wherein at least one of said allogeneic tumor cells is genetically modified to express CD80 (B7.1).

50. The method of claim 49, wherein said genetically modified allogeneic tumor is SW620 or COLO 205.

51. The method of claim 50, wherein said one or more allogeneic tumor cells is a combination of SW620, COLO 205, and SW403.

52. A method of stimulating an immune response in a patient having colorectal cancer, comprising administering to said patient a composition comprising a SW620 cell, a COLO 205 cell, and a SW403 cell, said SW620 and COLO 205 cells genetically modified to express CD80 (B7.1), and an allogeneic fibroblast cell genetically modified to express IL-2, whereby said allogeneic cell stimulates an immune response to an autologous tumor cell in said patient.
